(19) United States
(12) Patent Application Publication
(10) Pub. No.: US 2023/0036340 A1
(43) Pub. Date: Feb. 2, 2023

(54) TISSUE ENGINEERED VASCULAR GRAFTS WITH ADVANCED MECHANICAL STRENGTH

(71) Applicant: YALE UNIVERSITY, New Haven, CT (US)

(72) Inventors: Yibing Qyang, Guilford, CT (US); Laura Niklason, Greenwich, CT (US); Jiesi Luo, New Haven, CT (US); Lingfeng Qin, Hamden, CT (US); Liqiong Gui, Irvine, CA (US); Matthew Ellis, Issaquah, WA (US)

(21) Appl. No.: 17/781,478

(22) PCT Filed: Dec. 4, 2020
(86) PCT No.: PCT/US2020/063226

§ 371 Date: Jun. 1, 2022

Related U.S. Application Data
(60) Provisional application No. 62/943,577, filed on Dec. 4, 2019.

(51) Int. Cl. A61L 27/38; A61K 35/44; A61L 27/18; A61L 27/54; A61L 27/58; C12N 5/071

(52) U.S. Cl. CPC A61L 27/3834; A61K 35/44; A61L 27/18; A61L 27/54; A61L 27/58; C12N 5/0691; A61F 2/062

(57) ABSTRACT

The present invention provides a tissue-engineering vascular graft (TEVG) comprising a biodegradable scaffold, and a plurality of stem cell-derived vascular smooth muscle cells (VSMCs), wherein the plurality of stem cell-derived VSMCs are seeded on the biodegradable synthetic polymer scaffold and are cultured under mechanical and biochemical stimulation.

### CROSS-REFERENCE TO RELATED APPLICATIONS

This application claims the benefit of priority under 35 U.S.C. § 119(e) to U.S. Provisional Patent Application No. 62/943,577, filed Dec. 4, 2019, the contents of which are incorporated by reference herein in their entirety.

### STATEMENT REGARDING FEDERALLY SPONSORED RESEARCH OR DEVELOPMENT

This invention was made with government support under grant number HL116705 awarded by the National Institutes of Health. The government has certain rights in the invention.

### BACKGROUND OF THE INVENTION

Mechanically robust vascular grafts are in urgent clinical demand for treating cardiovascular diseases or providing hemodialysis access. While autologous or synthetic vascular grafts are clinically employed, the lack of suitable native vessels from patients or the potential risk of thrombosis and infection from synthetic materials hampers their application and efficacy (Akoh and Patel, 2010, J Vasc Access 11, 155-158; Conte, 2013, J Vasc Surg 57, 8S-13S). The application of human allograft vessels from cadavers (Madden et al., 2005, Annals of vascular surgery 19, 686-691) has also been reported. However, potential aneurysm, calcification, and thrombosis hinder their wide-spread clinical utilization. Tissue-engineered vascular grafts (TEVGs) provide an alternative resource of vascular grafts for patients who require arterial bypass or hemodialysis access. TEVGs with remarkable mechanical strength have been generated from human primary vascular smooth muscle cells (VSMCs) or fibroblasts (Dahl et al., 2011, Sci Transl Med 3, 68ra69; McAllister et al., 2009, Lancet 373, 1440-1446; Syedain et al., 2017, Sci Transl Med 9, eaan4209). To date, TEVGs from primary VSMCs coupled with decellularization have achieved promising results for hemodialysis access in clinical trials (Lawson et al., 2016, Lancet 387, 2026-2034). Acellular TEVGs therefore offer a readily available option for emergent vascular intervention (Elliott et al., 2019, Proc Natl Acad Sci USA 116, 12710-12719; Lawson et al., 2016, Lancet 387, 2026-2034; Wu et al., 2012, Nat Med 18, 1148-1153), but should also allow for effective ingrowth and vascular remodeling for long-term engraftment and ultimate replacement of the implanted graft by host vascular tissue. However, vascular cells from a substantial number of patients who may need TEVGs could have defective proliferation or vascular remodeling due to advanced age or diseases such as diabetes (Poh et al., 2005 Lancet 365, 2122-2124; Spinetti et al., 2008, Cardiovasc Res 78, 265-273), and as a result may not benefit from acellular TEVGs. As such, current acellular TEVGs may not work efficaciously for a considerable patient population in need. Accordingly, TEVGs engineered with non-immunogenic cells that retain the mechanical properties of native vascular tissues are needed. The present invention addresses this need.

### SUMMARY OF THE INVENTION

In certain embodiments, the present invention provides a tissue-engineering vascular graft (TEVG). The TEVG includes a biodegradable scaffold, and a plurality of stem cell-derived vascular smooth muscle cells (VSMCs), wherein the plurality of stem cell-derived VSMCs are seeded on the biodegradable synthetic polymer scaffold and are cultured under mechanical and biochemical stimulation. In some embodiments, the biodegradable scaffold includes one or more synthetic polymers selected from: polyglycolic acid (PGA), polylactic acid (PLA), polycaprolactone, polyethylene glycol, polylactic-co-glycolic acid (PLGA), poly(glycerol sebacate) (PGS), fast-degrading polymers, and/or combinations thereof. In some embodiments, the stem cell-derived VSMCs are derived from human induced pluripotent stem cells (hiPSCs) that are induced to differentiate into VSMCs. In some embodiments, the stem cell-derived VSMCs are allogeneic. In some embodiments, the hiPSCs are immunocompatible pluripotent stem cells. In some embodiments, the fast-degrading polymers include 87% glycolide, 7% trimethylene carbonate (TMC), and 6% polyethylene glycol.

In various embodiments, the TEVG further includes a plurality of stem cell-derived vascular endothelial cells (ECs). In some embodiments, the stem cells are hiPSCs. In some embodiments, the stem cell-derived ECs are allogeneic. In some embodiments, the hiPSCs are immunocompatible pluripotent stem cells.

In some embodiments, the mechanical stimulation includes incremental radial stretching and pulsatile radial distension. In some embodiments, the pulsatile radial distension has a pulse rate of about 110 to about 120 bpm.

In some embodiments, the biochemical stimulation includes TEVG culture media. In some embodiments, the TEVG culture media comprising transforming growth factor-β1 (TGF-β1) and does not include platelet-derived growth factor-BB (PDGF-BB).

In certain embodiments, the present invention provides a method of generating a tissue-engineered vascular graft (TEVG) as described herein, the method comprising: a) obtaining a plurality of hiPSCs; b) inducing the plurality of hiPSCs to differentiate into a population of hiPSC-VSMCs; c) seeding the population of hiPSC-VSMCs onto a biodegradable scaffold; and d) culturing the population of hiPSC-VSMCs on the biodegradable scaffold under mechanical and biochemical stimulation for a duration of time, thereby generating a hiPSC-TEVG. In some embodiments, the hiPSCs are allogeneic. In some embodiments, the hiPSCs are autogeneic.

In some embodiments, the biodegradable scaffold includes one or more synthetic polymers selected from: polyglycolic acid (PGA), polylactic acid (PLA), polycaprolactone, polyethylene glycol, polylactic-co-glycolic acid (PLGA), poly(glycerol sebacate) (PGS), fast-degrading polymers, and combinations thereof. In some embodiments, the fast-degrading polymer includes 87% glycolide, 7% trimethylene carbonate (TMC), and 6% polyethylene glycol.

In some embodiments, the mechanical stimulation includes incremental radial stretching and pulsatile radial distension. In some embodiments, the pulsatile radial distension has a pulse rate of about 110 to about 120 bpm. In some embodiments, the biochemical stimulation includes TEVG culture media. In some embodiments, the TEVG culture media comprising transforming growth factor-β1 (TGF-β1) and does not include platelet-derived growth factor-BB (PDGF-BB). In some embodiments, the population of hiPSC-VSMCs are cultured in media comprising one or more polyphenol. In some embodiments, the one or more polyphenols include epigallocatechin gallate (EGCG).

In various embodiments, the method further includes the intermediate step: b′) inducing the plurality of hiPSCs to differentiate into a population of hiPSC-derived ECs (hiPSC-EC).

In some embodiments, the method still further includes the step of: e) seeding the hiPSC-TEVG with the population of hiPSC-ECs, thereby endothelializing the TEVG. In some embodiments, the method further includes the intermediate step of: a′) modulating the human leukocyte antigen (HLA) expression of the plurality hiPSCs.

In certain embodiments, the present invention provides a tissue-engineering vascular graft (TEVG) comprising: a biodegradable scaffold, and a plurality of stem cell-derived vascular smooth muscle cells (VSMCs), and a plurality of stem cell-derived vascular endothelial cells (ECs), wherein the plurality of stem cell-derived VSMCs are seeded on the biodegradable synthetic polymer scaffold and are cultured under mechanical and biochemical stimulation. In some embodiments, the stem cell-derived ECs are allogeneic. In some embodiment, the stem cell-derived ECs are B2M⁻/CIITA⁻/CD47⁺ hiPSC-derived ECs (hiPSC-ECs).

### BRIEF DESCRIPTION OF THE DRAWINGS

FIG. 1 depicts exemplary strategies for generating human induced pluripotent stem cell (hiPSC)-based tissue engineered vascular grafts (hiPSC-TEVGs). Panel A depicts an exemplary current generation of hiPSC-TEVGs. Unlimited numbers of functional hiPSC-derived VSMCs (hiPSC-VSMCs), seeded onto a biodegradable polyglycolic acid (PGA) scaffold, and cultured in a bioreactor under novel incremental, radial distension for 8 weeks. The scaffold degrades as hiPSC-VSMCs produce extracellular matrix (ECM) proteins to form a hiPSC-TEVG with advanced mechanical strength. Panel B depicts an exemplary future strategy for hiPSC-TEVGs as an allogeneic cell-based therapy, as contemplated herein. An allogeneic human leukocyte antigen (HLA)-engineered, universal iPSC line could be used for large-scale production of VSMCs and endothelial cells (ECs) for cryopreservation for graft (≥6 mm or 2-4 mm) engineering and luminal endothelization. Cell-based hiPSC-TEVGs could be used to treat patients with defective vascular remodeling (e.g. advanced age or diabetes), coupled with moderate immune modulation and possible control of inflammation and/or blood glucose. Panel C depicts an exemplary future strategy for hiPSC-TEVGs as an acellular therapy. An allogeneic universal iPSC line is not required for VSMC production for TEVG engineering in this approach. VSMCs in the hiPSC-TEVGs could be decellularized, and acellular TEVGs with ECM could be stored at 4° C. for long-term use. Large diameter (≥6 mm) TEVGs could be immediately available for vascular intervention without the need for endothelialization (e.g. above the knee or hemodialysis). For small-diameter (2-4 mm) bypass applications (e.g. coronary artery or below the knee), the allogeneic, universal hiPSC-ECs would be used to coat the acellular grafts to prevent coagulation and luminal stenosis before implantation. Note that short-term storage and multi-site coordination for graft production and usage based on estimated clinical need are feasible and necessary for cell-containing TEVG applications in Panels B and C.

FIGS. 2A to 2G depict optimization of TEVG medium by generating engineered tissues from culturing hiPSC-VSMCs on biodegradable polyglycolic acid scaffolds. FIG. 2A depicts a schematic illustration of the method for developing tissue patches from “primed” hiPSC-VSMCs grown on biodegradable PGA scaffolds. The engineered tissues were cultured in TEVG media of one of four formulas: (1) supplemented with both transforming growth factor-β1 (TGF-β1) and platelet-derived growth factor-BB (PDGF-BB) (T/P), (2) TGF-β1 (T/−), (3) PDGF-BB (−/P), or (4) no growth factor (−/−) for 3 weeks. FIGS. 2B and 2C depict H&E staining and Masson's Trichrome staining of the engineered tissues derived from hiPSC-VSMCs seeded onto PGA scaffold. Red arrow heads indicate PGA remnants. Scale bars: 500 FIG. 2D depicts collagen weight per mesh of engineered tissue via hydroxyproline assay (One-way ANOVA with Tukey's multiple comparisons test; Mean values and S.E.M indicated by the error bars are shown; n=3; *: p<0.05; **: p<0.01; N.S: not significant). FIG. 2E depicts immunostaining of the engineered tissues from hiPSC-VSMCs. The sections were stained for VSMC markers MYH11 and α-SMA. DNA (nuclear) was counterstained by DAPI. White arrow heads indicate PGA remnants. Scale bar: 100 μm. FIG. 2F depicts TUNEL staining of the engineered tissues from hiPSC-VSMCs. DNA (nuclear) was counterstained by DAPI. White arrow heads indicate the TUNEL-positive apoptotic cells. Scale bar: 100 FIG. 2G depicts quantification of percentage of TUNEL-positive cells in engineered tissues (One-way ANOVA with Tukey's multiple comparisons test; Mean values and S.E.M indicated by the error bars are shown; n=3; **: p<0.01; N.S: not significant).

FIGS. 3A to 3P depict generation and characterization of tissue engineered vascular grafts from hiPSC-derived VSMCs under cyclic stretch in a bioreactor with pulsatile flow. FIG. 3A depicts a schematic illustration of the approach for generation and implantation of small caliber (3.2 mm of inner diameter) hiPSC-TEVGs. FIG. 3B depicts the rupture pressures of hiPSC-TEVGs developed after eight weeks of culture in the presence of pulsatile radial stress with 1% ultimate strain at different rates of 110-120, 150-160 and 190-200 beats per minute (One-way ANOVA with Tukey's multiple comparisons test; Mean values and S.E.M indicated by the error bars are shown; Filled dots, squares and triangles indicate the values of rupture pressure for individual hiPSC-TEVGs; n=3; **: p<0.01; ****: p<0.0001). FIGS. 3C-3F demonstrate generation of hiPSC-TEVGs with eight weeks of culture in the presence of pulsatile radial stress with 3% ultimate strain at 120 bpm. FIG. 3C depicts representative images of hiPSC-TEVGs after eight weeks of culture. Scale bar: 1 cm; (FIGS. 3D-3F) The wall thickness (FIG. 3D), rupture pressure (FIG. 3E) and suture retention strength (FIG. 3F) of hiPSC-TEVGs and native human umbilical arteries HUAs (Two-tailed unpaired Student's T-test was performed in FIGS. 3D-3F; Mean values and S.E.M indicated by the error bars are shown; Dots indicate the values of results for individual hiPSC-TEVGs or HUAs; n=4; N.S: not significant; *: p<0.05). FIGS. 3G-3J depict Histological examination (H&E staining (FIG. 3G), Masson's trichrome staining (FIG. 3H), Alizarin Red staining (FIG. 3I) and EVG staining (FIG. 3J) of hiPSC-TEVGs were performed. Asterisk indicates the lumen of the graft. Scale bar: 100 μm. FIGS. 3K-3N illustrates immunostaining of sections of hiPSC-TEVGs (FIGS. 3K-3M). Sections were stained for VSMC markers α-SMA, CNN1, and MYH11. DNA (nuclear) was counterstained by DAPI. Asterisk indicates the lumen of the graft. Scale bar: 100 μm. FIG. 3N depicts quantification of fluorescence intensity (gray value) of α-SMA, CNN1, and MYH11 per cell from three representative sections (>100 cells/section) in each hiPSC-TEVG (Mean values and S.E.M indicated by the error bars are shown; n=3). FIGS. 3O-3P depict immunostaining of KI67 in hiPSC-TEVGs (FIG. 3O). DNA (nuclear) was counterstained by DAPI. Asterisk indicates the lumen of the graft. Scale bar: 100 μm; FIG. 3P depicts quantification of percentage of KI67-positive cells from three representative sections (>100 cells/section) in each hiPSC-TEVG (Mean values and S.E.M indicated by the error bars are shown; n=3).

FIGS. 4A to 4S demonstrate characterization of hiPSC-TEVGs in nude rats 30 days post-operation. FIG. 4A depicts a schematic illustration of an exemplary experimental design for implantation of hiPSC-TEVGs into nude rats and following ultrasonographic analysis. Six hiPSC-TEVGs (TEVG1-6) were independently implanted into six nude rats as interpositional aortic grafts. FIG. 4B depicts a representative image of explanted TEVG graft (TEVG2) on day 30 post-operation. The dashed line indicates the position of sectioning. Scale bar: 5 cm. FIG. 4C depicts the inner diameters, outer diameters and length of the six implanted hiPSC-TEVGs over time (evaluated weekly) during 30 days of implantation by ultrasonographic analysis. FIG. 4D depicts wall thickness of hiPSC-TEVGs during 30 days of implantation by ultrasonographic analysis (Repeated Measures one-way ANOVA with Tukey's multiple comparisons test; Mean values and S.E.M are indicated by the dots and error bars; n=6; N.S: not significant). Note that wall thickness of each TEVG during the four-week implantation was normalized to the average wall thickness of TEVGs (n=6) one-week post-implantation. FIG. 4E depicts representative stress-strain plots of tissue rings sectioned from pre-implant or explanted hiPSC-TEVGs. In FIG. 4F, mechanical parameters, including maximum modulus, ultimate tensile stress and failure strain were compared between the tissue rings sectioned from pre-implant or explanted hiPSC-TEVGs (Two-tailed paired Student's T-test; Mean values and S.E.M indicated by the error bars are shown; n=3; N.S: not significant). FIGS. 4G-4J illustrate histological analysis (H&E staining in FIG. 4G; Masson's trichrome staining in FIG. 4H; Alizarin Red staining in FIG. 4I; and EVG staining in FIG. 4J) of explanted hiPSC-TEVGs (TEVG2). Asterisk indicates the lumen of the graft. Scale bar: 100 μm. FIGS. 4K-4S illustrate immunostaining of sections of hiPSC-TEVGs. The sections were stained for VSMC markers α-SMA (FIG. 4K, K′, K″), CNN1 (FIG. 4L, L′, L″), and MYH11 (FIG. 4M, M′, M″), and fluorescence intensity (gray value) of α-SMA, CNN1, and MYH11 per HLA-A-positive cell from three representative sections (>80 cells/section) in each hiPSC-TEVG was quantified, as shown in FIG. 4N (Mean values and S.E.M indicated by the error bars are shown; n=3). Cell proliferation marker KI67 was stained, shown in FIG. 4O, and percentage of KI67-positive cells in HLA-A-positive cells in three representative sections (>80 cells/section) in each hiPSC-TEVG was quantified, shown in FIG. 4P (Mean values and S.E.M indicated by the error bars are shown; n=3). Additionally, sections were stained for macrophage marker CD68 (Q) (positive cells indicated by white arrow heads) and endothelial cell markers CD31 (FIG. 4R) and vWF (FIG. 4S). The human specific surface antigen (HLA-A) was stained to indicate the human cells in TEVGs, shown FIGS. 4K-4M. The white dashed line indicates the boundary between the human cells and host rat cells (shown in FIGS. 4K, 4L, 4M and 4O). DNA (nuclear) was counterstained by DAPI. Asterisk indicates the lumen of the graft. Scale bar: 100 μm.

FIGS. 5A to 5N depict generation and characterization of human induced pluripotent stem cell-derived vascular smooth muscle cells. FIG. 5A depicts a schematic illustration of VSMC differentiation of hiPSCs using an embryoid body (EB)-based approach. Briefly, floating EBs were generated by resuspending ethylenediaminetetraacetic acid (EDTA, 0.5 mM) dissociated single hiPSCs in media gradually transitioned from mTeSR1 to EB differentiation medium in a 6-day low attachment suspension culture. EBs were then seeded on a gelatin-coated culture dish for six days with EB differentiation medium. The adherent EB-derived cells were next cultured in SmGM-2 VSMC growth medium for 7-10 days, resulting in the production of hiPSC-VSMCs in proliferation state (hiPSC-VSMCs-P). The hiPSC-VSMCs in mature state (hiPSC-VSMCs-M) were derived by culturing hiPSC-VSMCs-P in a maturation medium containing 1% FBS and 1 ng/ml TGF-β1 for seven days. FIG. 5B demonstrates approaches for deriving EBs from hiPSCs. Representative images of EBs derived from hiPSCs on day 0, 2 and 4 of differentiation, generated via either the current EDTA-mediated dissociation of hiPSC colonies coupled with a 4-day medium transition from the mTeSR to the EB differentiation medium, or the previous approach which included dispase-mediated dissociation of hiPSC colonies coupled with a 2-day medium transition. Scale bar: 200 FIG. 5C illustrates immunostaining of VSMC (α-SMA, CNN1 and MYH11), ECM (COL1 and ELN) and pluripotency (OCT4) markers in hiPSC-VSMCs-P, hiPSC-VSMCs-M, primary VSMCs-P, primary VSMCs-M, and undifferentiated hiPSCs. DNA (nuclear) was counterstained by DAPI. Scale bar: 200 FIG. 5D depicts the percentage of cells (hiPSC-VSMCs-P, hiPSC-VSMCs-M, primary VSMCs-P, primary VSMCs-M, and undifferentiated hiPSCs) positive for VSMC (α-SMA, CNN1 and MYH11), ECM (COL1 and ELN) and pluripotency (OCT4) markers from immunostaining (One-way ANOVA with Tukey's multiple comparisons test; Mean values and S.E.M indicated by the error bars are shown; n=3; ***: p<0.001; ****: p<0.0001; N.S: not significant). FIG. 5E depicts quantification of fluorescence intensity of α-SMA, CNN1, MYH11, COL1, ELN and OCT4 in hiPSC-VSMCs-P, hiPSC-VSMCs-M, primary VSMCs-P, primary VSMCs-M and undifferentiated hiPSCs. Three independent experiments were completed for each cell type, and 100 cells or above were quantified in each experiment. Values in y-axis represent fold changes of average fluorescence intensity (gray value) per cell relative to that of undifferentiated hiPSCs (One-way ANOVA with Tukey's multiple comparisons test; Mean values and S.E.M indicated by the error bars are shown; n=3; *: p<0.05; **: p<0.01; ***: p<0.001; ****: p<0.0001; N.S: not significant). FIG. 5F depicts contractility of hiPSC-VSMCs-P, primary VSMCs-P, hiPSC-VSMCs-M, and primary VSMCs-M in response to treatment (before and after 20 minutes) of 1 mM carbachol. Representative cells, both basal and contracted, were indicated by the lines before and after carbachol treatment, respectively. Scale bar: 200 μm. FIG. 5G depicts quantification of reduced cell area of hiPSC-VSMCs and human primary VSMCs in proliferation and maturation medium in response to 1 mM carbachol or vehicle control (PBS) (One-way ANOVA with Tukey's multiple comparisons test; Mean values and S.E.M indicated by the error bars are shown; n=3; *: p<0.05; **: p<0.01; ****: p<0.0001; N.S: not significant). FIG. 5H depicts a schematic illustration of derivation of hiPSC-VSMCs via previously established approach (Gen 1 hiPSC-VSMCs), or currently optimized approach with expansion in SmGM medium (Gen 2 hiPSC-VSMCs-SmGM or hiPSC-VSMCs-P) or DMEM containing 10% FBS (Gen 2 hiPSC-VSMCs-10% FBS, or primed hiPSC-VSMCs). FIG. 5I depicts results from a 3-(4,5-dimethylthiazol-2-yl)-2,5-diphenyltetrazolium bromide (MTT) assay that was performed to monitor the proliferation of Gen 1 hiPSC-VSMCs, Gen 2 hiPSC-VSMCs-SmGM, or Gen 2 hiPSC-VSMCs-10% FBS (One-way ANOVA with Tukey's multiple comparisons test; Mean values and S.E.M indicated by the error bars are shown; n=3; ***: p<0.001; ****: p<0.0001; N.S: not significant). FIG. 5J depicts qRT-PCR analysis of relative mRNA transcript amounts of VSMC (α-SMA, CNN1, MYH11 and SMTH) and ECM (COL1, COL3 and ELN) markers in Gen 1 hiPSC-VSMCs, Gen 2 hiPSC-VSMCs-SmGM, Gen 2 hiPSC-VSMCs-10% FBS, or the control HUVECs for seven days. Values in the y axis represent fold changes relative to human GAPDH expression. Gene expression in hiPSC-VSMCs was normalized to that of in HUVECs (One-way ANOVA with Tukey's multiple comparisons test; Mean values and S.E.M indicated by the error bars are shown; n=4; *: p<0.05; **: p<0.01; ***: p<0.001; ****: p<0.0001; N.S: not significant). FIG. 5K depicts an MTT assay indicating the proliferation capability of neuroectodermal (NE), lateral plate mesodermal (LM) and paraxial mesodermal (PM) hiPSC-VSMCs and hiPSC-VSMCs derived from EB-based approach (One-way ANOVA with Tukey's multiple comparisons test; Mean values and S.E.M indicated by the error bars are shown; n=3; **: p<0.01; ****: p<0.0001). FIG. 5L depicts quantification of fluorescence intensity of α-SMA and MYH11 in hiPSC-VSMCs in engineered vascular tissues cultured in different TEVG media (T/P, T/−, −/P and −/−). Values in the y axis represent fold changes of average fluorescence intensity (gray value) per cell relative to that of hiPSC-VSMCs in vascular tissue cultured in −/− medium. Three independent vascular tissue constructs were generated and immunostained, and at least 90 cells were analyzed for each group in each batch (One-way ANOVA with Tukey's multiple comparisons test; Mean values and S.E.M indicated by the error bars are shown; n=3; *: p<0.05; **: p<0.01; ***: p<0.001; N.S: not significant). FIG. 5M depicts contractility of hiPSC-VSMCs (primed) in the TGF-β1-containing TEVG medium (T/−) in response to the treatment (before and after 20 minutes) of 1 mM carbachol. Representative cells were indicated by the blue (basal) and red (contracted) lines before and after carbachol treatment, respectively. Scale bar: 200 μm. FIG. 5N depicts quantification of reduced cell area of hiPSC-VSMCs (primed) in TEVG medium (T/−) in response to 1 mM carbachol or vehicle control (PBS) (Two-tailed unpaired Student's T-test; Mean values and S.E.M indicated by the error bars are shown; n=3; ***: p<0.001).

FIGS. 6A to 6F demonstrate the characterization of hiPSC-VSMCs cultured under static or stretched condition in the previously reported or currently optimized TEVG medium. FIG. 6A depicts qRT-PCR analysis of relative mRNA transcript amounts of VSMC (α-SMA and CNN1), extracellular matrix (COL1, COL3 and ELN), focal adhesion (vinculin), adherens junction (N-cadherin), and metabolism-associated (GLUT1, GLUT4, CS, and PGC1α) markers in hiPSC-VSMCs, which were cultured in the previously reported TEVG medium (T/P) or currently optimized TEVG medium (T/−) under static or stretched (uniaxial cyclic stretching: 2.5% distention at 2.75 Hz) conditions with a FLEXCELL™ FX-6000T™ Tension System for 2 days, respectively. Y-axis values represent fold changes relative to human GAPDH expression. The gene expression in hiPSC-VSMCs of experimental groups is normalized to that of hiPSC-VSMCs cultured in the absence of stretching with the previously reported TEVG medium (static, T/P). Interaction between the effect of medium and mechanical treatment on gene expression was evaluated via two-way ANOVA with Tukey's multiple comparisons test to assess differences among independent groups. Gene expression was synergistically upregulated by optimized culture medium (T/−) and stretching for expression of α-SMA (interaction p=0.0005), CNN1 (interaction p=0.001), COL1 (interaction p=0.01), and COL3 (interaction p=0.04). Expression of ELN was independently upregulated by optimized culture medium (T/−) and stretching (p=0.003 for culture medium and p<0.0001 for stretching; interaction between factors was not significant, p=0.2). Additionally, stretching alone increased the expression of vinculin, N-cadherin, GLUT1, GLUT4, CS, and PGC1α independent of culture medium utilized. Mean values and S.E.M indicated by the error bars are shown; n=3; *: p<0.05; **: p<0.01; ***: p<0.001; ****: p<0.0001; N. S: not significant. FIG. 6B illustrates immunostaining of ECM markers (collagen type 1 and elastin), filamentous actin (phalloidin), and cell-cell adhesion marker (N-cadherin) in hiPSC-VSMCs cultured in the previously reported (T/P) or currently optimized (T/−) TEVG medium under static or stretched conditions. White double arrows show stretch directionality. DNA (nuclear) was counterstained by DAPI. Scale bar: 200 FIG. 6C depicts quantification of collagen type I, elastin, phalloidin, and N-cadherin staining intensity relative to cell number in hiPSC-VSMCs under static or stretched conditions with the previously reported (T/P) or currently optimized (T/−) TEVG medium. Values in the y axis represent fold change of fluorescence intensity (gray value) per cell relative to that of cells cultured in the T/P medium under a static condition (static, T/P). Two-way ANOVA showed that optimized culture medium (T/−) and mechanical stretching synergistically upregulated the expression of collagen I (interaction p<0.0001) and filamentous actin bundles (phalloidin) (interaction p=0.03). Expression of ELN was upregulated by optimized culture medium (T/−) and stretching independently. Additionally, stretching alone increased the expression of N-cadherin independent of culture medium utilized. Three independent experiments were completed for the experimental groups, and 100 cells or above were quantified for each group in each experiment. Mean values and S.E.M indicated by the error bars are shown; n=3; *: p<0.05; **: p<0.01; ***: p<0.001; ****: p<0.0001; N.S: not significant. FIG. 6D depicts collagen weight per hiPSC-VSMC cultured in the previously reported (T/P) or currently optimized (T/−) TEVG medium under static or stretched conditions via a hydroxyproline assay. Two-way ANOVA showed that optimized culture medium (T/−) and mechanical stretching promoted collagen deposition independently. Mean values and S.E.M indicated by the error bars are shown; n=3; *: p<0.05; ****: p<0.0001. FIG. 6E depicts glucose consumption rates of hiPSC-VSMCs cultured in the previously reported (T/P) or currently optimized (T/−) TEVG medium under static or stretched conditions. Two-way ANOVA showed that stretching alone increased glucose consumption independent of culture medium utilized. Mean values and S.E.M indicated by the error bars are shown; n=3; ***: p<0.001. FIG. 6F depicts the cellular ATP concentration of hiPSC-VSMCs cultured in the previously reported (T/P) or currently optimized (T/−) TEVG medium under static or stretched conditions. Two-way ANOVA showed that optimized culture medium (T/−) and mechanical stretching synergistically increased cellular ATP production (interaction p=0.01). Mean values and S.E.M indicated by the error bars are shown; n=3; **: p<0.01, ***: p<0.001, ****: p<0.0001; N.S: not significant.

FIGS. 7A to 7R depict histological characterization and physical properties of human umbilical arteries, porcine coronary arteries and hiPSC-TEVGs. FIGS. 7A to 7D illustrate histological examination (H&E staining (FIG. 7A); Masson's trichrome staining (FIG. 7B), Alizarin Red staining (FIG. 7C); and EVG staining (FIG. 7D)) of HUAs that were performed. Asterisk indicates the lumen of the graft. Scale bar: 100 μm. FIGS. 7E to 7H illustrate immunostaining of sections of HUAs (FIGS. 7E-7G). Sections were stained for VSMC markers α-SMA, CNN1, and MYH11. DNA (nuclear) was counterstained by DAPI. Asterisk indicates the lumen of the graft. Scale bar: 100 μm; FIG. 7H depicts the quantification of fluorescence intensity (gray value) of α-SMA, CNN1, and MYH11 per cell in three representative sections (>100 cells/section) in each HUA (Mean values and S.E.M indicated by the error bars are shown; n=3). FIGS. 7I to 7J illustrate immunostaining of KI67 in HUAs (FIG. 7I). DNA (nuclear) was counterstained by DAPI. Asterisk indicates the lumen of the graft. Scale bar: 100 μm; FIG. 7J depicts quantification of percentage of KI67-positive cells from three representative sections (>100 cells/section) in each HUA (Mean values and S.E.M indicated by the error bars are shown; n=3). FIGS. 7K to 7O depict a representative photograph of porcine coronary arteries (FIG. 7K), a representative stress-strain plot (FIG. 7L), and mechanical parameters, including maximum modulus (FIG. 7M), ultimate tensile stress (FIG. 7N) and failure strain (FIG. 7O) of the tissue rings sectioned from porcine coronary arteries (Mean values and S.E.M indicated by the error bars are shown in FIGS. 7M-7O; Dots indicate the values for individual porcine coronary arteries; n=3). FIG. 7P depicts a representative photograph of a tissue ring sectioned from a hiPSC-TEVG mounted onto micromanipulators and immersed in the temperature-controlled perfusion bath containing Tyrode's solution for contractility assay. Arrows to the right indicate the tissue ring, and arrows to the left indicate the arm of the force transducer. FIG. 7Q depicts contractility changes (Pascal) of tissue rings sectioned from pre-implant or explanted hiPSC-TEVGs in response to the vasoconstrictor carbachol (1 mM) for 30 min (Two-tailed unpaired Student's T-test; Mean values and S.E.M indicated by the error bars are shown; n=3; N.S: not significant). FIG. 7R depicts the percentage of human cells (HLA-A+ cells) positive for VSMC markers (α-SMA, CNN1 or MYH11) in hiPSC-TEVGs 30 days after implantation. The sections were co-stained for HLA-A and α-SMA, CNN1, or MYH11 from three representative sections (>80 cells/section) in each hiPSC-TEVG (Mean values and S.E.M indicated by the error bars are shown; n=3).

FIGS. 8A-8Q depict generation of engineered tissue using a gene-edited hiPSC line and characterization of hiPSC-TEVGs in a nude rat model 60 Days post-operation. FIG. 8A is a schematic illustration of an exemplary strategy of generating HLA-C-retained hiPSCs (Xu et al., 2019, Cell Stem Cell 24, 566-578 e567.). FIG. 8B illustrates immunostaining of VSMC (α-SMA, CNN1 and MYH11), ECM (COL1 and ELN), pluripotency (OCT4), and human surface antigen (HLA-A) markers in HLA-C-retained hiPSC-VSMCs in proliferation medium (HLA-C-retained hiPSC-VSMCs-P) or maturation medium (HLA-C-retained hiPSC-VSMCs-M). Wild type hiPSC-VSMCs were stained for HLA-A as positive control. DNA (nuclear) was counterstained by DAPI. Scale bar: 200 μm. FIG. 8C depicts contractility of HLA-C-retained hiPSC-VSMCs in proliferation medium (HLA-C-retained hiPSC-VSMCs-P) or maturation medium (HLA-C-retained hiPSC-VSMCs-M) in response to treatment (before and after 20 minutes) of 1 mM carbachol. Representative cells were indicated by the blue (basal) and red (contracted) lines before and after carbachol treatment, respectively. Scale bar: 200 μm. FIG. 8D depicts quantification of reduced cell area of HLA-C-retained hiPSC-VSMCs in proliferation medium (HLA-C-retained hiPSC-VSMCs-P) or maturation medium (HLA-C-retained hiPSC-VSMCs-M) in response to 1 mM carbachol or vehicle control (PBS) (One-way ANOVA with Tukey's multiple comparisons test; Mean values and S.E.M indicated by the error bars are shown; n=3; *: p<0.05; ***: p<0.001). FIGS. 8E to 8H illustrate H&E staining (FIGS. 8E and 8F) and Masson's Trichrome (FIGS. 8G and 8H) staining of the engineered tissues derived from “primed” HLA-C-retained hiPSC-VSMCs seeded onto PGA scaffolds and cultured for three weeks. Arrow heads in both FIG. 8F and FIG. 8H indicate PGA remnants. Scale bars: 500 (FIGS. 8E and 8G) and 100 (FIGS. 8F and 8H)μm. FIG. 8I illustrates immunostaining of the engineered tissues from hiPSC-VSMCs. Section of engineered tissues derived from HLA-C-retained hiPSC-VSMCs was stained for α-SMA and MYH11. DNA (nuclear) was counterstained by DAPI. White arrow heads indicate PGA remnants. Scale bar: 100 μm. FIG. 8J depicts collagen weight per mesh of engineered tissue generated using “primed” HLA-C-retained or wild type hiPSC-VSMCs via hydroxyproline assay (Two-tailed unpaired Student's T-test; Mean values and S.E.M indicated by the error bars are shown; n=3; N.S: not significant). FIG. 8K depicts a representative image of an explanted TEVG graft on day 60 post-implantation. The dashed line indicates the position of sectioning. FIGS. 8L to 8O depict Histological analysis (H&E staining (FIG. 8L), Masson's trichrome staining (FIG. 8M), Alizarin Red staining (FIG. 8N) and EVG staining (FIG. 8O)) of an explanted TEVG graft on day 60 post-implantation. Asterisk indicates the lumen of the graft. The dashed line suggests the boundary between the human cells and host rat cells (FIG. 8O). Arrows indicate limited, discontinuous, extracellular elastin fibers in the medial layers of hiPSC-TEVGs. Also note the existence of immature, disorganized ELN fibers below the dashed line potentially generated by rat host cells. Scale bar: 100 μm. FIGS. 8P and 8Q illustrate immunostaining of sections of explanted TEVG graft on day 60 post-implantation. The section was stained for VSMC marker α-SMA (Panels P, P′, P″), MYH11 (Panels Q, Q′, Q″) and human surface antigen HLA-A (pseudo-color). DNA (nuclear) was counterstained by DAPI. Asterisk indicates the lumen of the graft. The white dashed line indicates the boundary between the human cells and host rat cells. Scale bar: 100 μm.

FIG. 9 depicts an exemplary method of the present invention.

FIGS. 10A-10E depict the decellularization of TEVGs and endothelialization of grafts with hiPSC-derived endothelial cells (hiPSC-ECs). FIGS. 10A and 10B illustrate the generation of and staining of TEVGs with H&E, α-SMA, MYH11, HLA-A and DNA (DAPI). *indicate graft lumen, and white dashed lines demarcate graft border. FIG. 10C depicts a scheme for luminal coating of a decellularized vessel with ECs in a bioreactor. FIG. 10D depicts immunostaining of EC markers (CD31 and eNOS) of a section of a decellularized graft coated with ECs. FIG. 10E depicts the coverage of the lumen of decellularize vessel by hiPSC-ECs. EC coverage rates of three representative segments of two vessels were quantified based on CD31, eNOS staining. Scale bars: 10B (100 μm), 10D (500 μm).

FIGS. 11A-11G depicts the generation of universal hiPSC-ECs. FIG. 11A depicts a scheme of CRISPR-Cas9-mediated major histocompatibility complex (MHC) class I and II knock-out (KO) and ectopic expression of CD47 (“don't eat me” signal that inhibits phagocytosis (Jaiswal et al., 2009, Cell 138, 271-85; Deuse et al., 2019, Nat Biotechnol. 37, 252-258) via TALEN-mediated knock-in (KI) at AAVSI “safe harbor” gene locus in hiPSCs. Note that “CRISPR”, “Cas9”, and “TALEN” are the abbreviations of “clustered regularly interspaced short palindromic repeats”, “CRISPR associated protein 9”, and “transcription activator-like effector nucleases”, respectively. FIG. 11B depicts immunostaining of EC markers (VE-cadherin and CD31) in hiPSC-ECs. Scale bar 200 μm. FIG. 11C depicts MHC class I (HLA-A-B-C) and class II (HLA-DR) expression in hiPSC-ECs and primary human umbilical vein endothelial cell (HUVECs) analyzed through flow cytometry. FIG. 11D depicts CD47 expression measured via flow cytometry in universal hiPSC-ECs and control cells. FIGS. 11E-11F depict interferon gamma (IFN-γ) enzyme-linked immunosorbent assay (ELISA) results from supernatants in 3-day co-cultures of human CD4+ and CD8+ effector memory T-cells with universal or unedited hiPSC-ECs (run in triplicate in an experiment). FIG. 11G depicts carboxyfluorescein succinimidyl ester (CFSE) dilution and HLA-DR activation in 7-day co-cultures of CD4+ T cells with various EC types (run in triplicate in an experiment).

FIGS. 12A-12B depicts polyphenol enhancement of elastin deposition by hiPSC-VSMCs. FIG. 12A depicts a schematic of polyphenol epigallocatechin gallate (EGCG) on enhancing elastin (ELN) deposition. FIG. 12B depicts results from immunostaining of phalloidin (cytoplasmic filamentous actin) and ELN in hiPSC-VSMCs with or without decellularization treated with polyphenol (10 μg/ml) or DMSO control in DMEM medium containing 3% FBS and 1 ng/ml TGF-β1 for 9 days. Prior to staining, cells were kept intact or decellularized with 40 mM ammonium hydroxide containing 0.5% Triton X-100. Nuclei: DAPI. Note that the complete decellularization and the signals of extracellular ELN in both cellularized and decellularized hiPSC-VSMCs treated with polyphenol. Scale bar: 200 μm.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, to “alleviate” a disease, defect, disorder or condition means reducing the severity of one or more symptoms of the disease, defect, disorder or condition.

As used herein, “autologous” refers to a biological material derived from the same individual into whom the material will later be re-introduced.

As used herein, “allogeneic” refers to a biological material derived from a genetically different individual of the same species as the individual into whom the material will be introduced.

As used here, “biocompatible” refers to any material, which, when implanted in a mammal, does not provoke a significant adverse response in the mammal. A biocompatible material, when introduced into an individual, is not toxic or injurious to that individual, nor does it induce immunological rejection of the material in the mammal.

As used herein, the terms “biocompatible polymer” and “biocompatibility” when used in relation to polymers are recognized in the art. For example, biocompatible polymers include polymers that are generally neither toxic to the host, nor degrade (if the polymer degrades) at a rate that produces monomeric or oligomeric subunits or other byproducts at toxic concentrations in the host. In one embodiment, biodegradation generally involves degradation of the polymer in a host, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in one embodiment, toxicology of a biodegradable polymer intended for in vivo use, such as implantation or injection into a patient, may be determined after one or more toxicity analyses. It is not necessary that any subject composition have a purity of 100% to be deemed biocompatible; indeed, it is only necessary that the subject compositions be biocompatible as set forth above. Hence, a subject composition may include polymers comprising 99%, 98%, 97%, 96%, 95%, 90%, 85%, 80%, 75% or even less of biocompatible polymers, e.g., including polymers and other materials and excipients described herein, and still be biocompatible.

As used herein, a “graft” refers to a composition that is implanted into an individual, typically to replace, correct or otherwise overcome a cell, tissue, or organ defect. A graft may include a scaffold. In certain embodiments, a graft includes decellularized tissue. In some embodiments, the graft may include a cell, tissue, or organ. The graft may consist of cells or tissue that originate from the same individual; this graft is referred to herein by the following interchangeable terms: “autograft,” “autologous transplant,” “autologous implant” and “autologous graft.” A graft comprising cells or tissue from a genetically different individual of the same species is referred to herein by the following interchangeable terms: “allograft,” “allogeneic transplant,” “allogeneic implant” and “allogeneic graft.” A graft from an individual to his identical twin is referred to herein as an “isograft,” a “syngeneic transplant,” a “syngeneic implant” or a “syngeneic graft.” A “xenograft,” “xenogeneic transplant” or “xenogeneic implant” refers to a graft from one individual to another of a different species.

As used herein, “scaffold” refers to a structure, comprising a biocompatible material that provides a surface suitable for adherence of a substance and proliferation of cells. A scaffold may further provide mechanical stability and support. A scaffold may be in a particular shape or form so as to influence or delimit a three-dimensional shape or form such as that assumed by a population of proliferating cells. Such shapes or forms include, but are not limited to, films (e.g. a form with two-dimensions substantially greater than the third dimension), ribbons, cords, sheets, flat discs, cylinders, spheres, three-dimensional amorphous shapes, etc.

As used herein, the terms “subject” and “patient” are used interchangeably. As used herein, a subject is preferably a mammal such as a non-primate (e.g., cows, pigs, horses, cats, dogs, rats, etc.) and a primate (e.g., monkey and human), most preferably a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides tissue-engineered vascular grafts (TEVG) and methods for generating TEVGs. The TEVGs as described herein can be generated with one or more populations of cell derivatives from human induced pluripotent stem cells (hiPSCs). The TEVGs of the present invention have advanced mechanical strength. The TEVGs of the present invention provide small caliber (2-4 mm inner diameter) vascular grafts.

### Tissue-Engineered Vascular Grafts

The present invention provides tissue-engineering vascular grafts (TEVGs): The TEVGs include one or more biodegradable scaffolds. The biodegradable scaffolds may include one or more synthetic polymers, one or more biopolymers, and or combinations thereof. The synthetic polymers can include one or more of polyglycolic acid (PGA), polylactic acid (PLA), polycaprolactone, polyethylene glycol, polylactic-co-glycolic acid (PLGA), poly(glycerol sebacate) (PGS), fast-degrading polymers such as that comprising 87% glycolide, 7% trimethylene carbonate (TMC), and 6% polyethylene glycol and/or combinations thereof.

The scaffolds may include one or more polymer meshes including for example, non-woven polymer meshes. The polymer mesh may have a thickness of 0.05 mm to about 0.1 mm, about 0.1 mm to about 0.3 mm, about 0.3 mm to about 0.5 mm, about 0.5 mm to about 0.7 mm, about 0.7 mm to about 0.9 mm, about 1 mm and the like. The polymer mesh may have a square shape with side lengths of about 1 mm to about 2 mm, about 2 mm to about 3 mm, about 3 mm to about 4 mm, about 4 mm to about 5 mm, about 5 mm to about 6 mm, about 6 mm to about 7 mm, about 7 mm to about 8 mm, about 8 mm to about 9 mm about 9 mm to about 10 mm and so on. The polymer meshes may be coated with one or more compounds for improving cell adhesion. For example, the polymer mesh may be coated with gelatin, poly-lysine, and the like. In some embodiments, the polymer mesh may be coated with one or more extracellular matrix proteins and/or fragments thereof. The extracellular matrix proteins may include one or more of gelatin, fibronectin, laminin, collagen, vitronectin, glycosaminoglycan, elastin, fibrillin, and/or combinations thereof.

The one or more scaffolds may be seeded with one or more populations of cells. The cells can include stem cells such as embryonic stem cells, mesenchymal stem cells, bone marrow-derived stem cells, hematopoietic stem cells, and the like. The cells can include somatic cells including vascular somatic cells such as smooth muscle cells, endothelial cells, fibroblast, and the like. The cells may include stem cell-derived vascular smooth muscle cells (VSMCs), stem cell-derived vascular endothelial cells (ECs), and/or combinations thereof. The cells can include somatic cell-derived stem cells such as, for example, human induced pluripotent stem cells (hiPSCs). In some embodiments, the cells include vascular cells induced from hiPSCs, including for example, hiPSC-derived vascular smooth muscle cells (hiPSC-VSMCs), hiPSC-derived vascular endothelial cells (hiPSC-ECs), and the like. The hiPSCs may include allogeneic stem cells, autogeneic stem cells, xenogeneic stem cells, gene-edited stem cells and/or combinations thereof.

The one or more populations of cells may include hiPSCs that are immunocompatible pluripotent stem cells. For example, the one or more populations of hiPSCs may have modulated or abrogated expression of one or more human leukocyte antigens (HLAs). In some embodiments, the one or more populations of hiPSCs have modulated expression of one or more of HLA-A alleles, HLA-B alleles, HLA-C alleles, one or more class II HLAs, and/or one or more combinations thereof.

The one or more populations of cells may be seeded onto the one or more biodegradable synthetic polymer scaffolds. The cells may be cultured under mechanical stimulation, biochemical stimulation, and/or combinations thereof. The mechanical stimulation may include

wherein the plurality of stem cell-derived VSMCs are seeded on the biodegradable synthetic polymer scaffold and are cultured under mechanical and biochemical stimulation. The mechanical stimulation may include incremental radial stretching, pulsatile radial distension, and or combinations thereof. The radial stretching may include incremental radial stretching. The incremental radial stretching may include radial strain having radial distension of up to about 0.5%, about 0.5% to about 1%, about 1% to about 1.5%, about 1.5% to about 2%, about 2% to about 2.5%, about 2.5% to about 3%, about 3% to about 3.5%, about 3.5% to about 4%, about 4% to about 4.5%, about 4.5% to about 5%, and/or greater than about 5%. In some embodiments, and incremental strain may be applied for a duration including up to about 1 day, about 1 day to about 1 week, about 1 week to about 2 weeks, about 2 weeks to about 3 weeks, about 3 weeks to about 4 weeks, about 4 weeks to about 5 weeks, about 5 weeks to about 6 weeks, about 6 weeks to about 7 weeks, about 7 weeks to about 8 weeks, about 8 weeks to about 9 weeks, about 9 weeks to about 10 weeks, or greater than about 10 weeks. The incremental strain may be gradually increased or decreased at a continuous rate over an increment of up to 1 week, about 1 week to about 2 weeks, about 2 weeks to about 3 weeks, about 3 weeks to about 4 weeks, or greater than 4 weeks. The pulsatile radial distension may include pulsatile radial distension having a pulse rate of up to about 110 bpm, about 110 bpm to about 120 bpm, about 120 bpm to about 130 bpm, about 130 bpm to about 140 bpm, about 140 bpm to about 150 bpm, about 150 bpm to about 160 bpm, about 160 to about 170 bpm, about 170 to about 180 bpm, about 180 to about 190 bpm, about 190 to about 200 bpm, and the like. The incremental strain and/or pulsatile radial distention may be applied as a regime including: 1) one or more phases having one or more particular radial strains (e.g., 0.5%, up to 5%, etc.) for one or more particular durations (e.g., 1 week, 4 weeks, etc.), 2) one or more particular pulsatile radial distensions (e.g., 110 bpm, 120 bpm, etc.) for one or more particular durations (e.g., 1 week, 4 weeks, etc.), and 3) one or more combinations thereof. Each phase may have a particular duration of about 1 day to about 1 week, about 1 week to about 2 weeks, about 2 weeks to about 3 weeks, about 3 weeks to about 4 weeks, about 4 weeks to about 5 weeks, about 5 weeks to about 6 weeks, about 6 weeks to about 7 weeks, about 7 weeks to about 8 weeks, and/or increments or combinations thereof. The regime may have a total duration of about 4 weeks, about 4 weeks to about 8 weeks, about 8 weeks to about 12 weeks, and/or increments thereof.

The biochemical stimulation may include culturing the one or more populations of cells in one or more culture media, including, for example, TEVG culture media as described elsewhere herein. The culture media may include or may exclude one or more growth factors and/or other molecules and reagents including for example, transforming growth factor-β1 (TGF β1), platelet-derived growth factor-BB (PDGF-BB), fibroblast growth factors (FGFs), bone morphogenetic factors (BMPs), vascular endothelial growth factors (VEGFs), latent TGF-beta binding proteins (LTBPs), epidermal growth factor (EGF), copper sulfate, ascorbic acid, retinoic acid, polyphenols (such as tannic acid, epigallocatechin gallate (EGCG) and pentagalloylglucose [PGG]), microRNA-29 inhibitors, proteoglycan inhibitors, fetal bovine sera, human platelet lysates, human sera, and or combinations thereof.

### Methods for Generating TEVGs

The present invention provides methods 900 for generating one or more TEVGs as described herein.

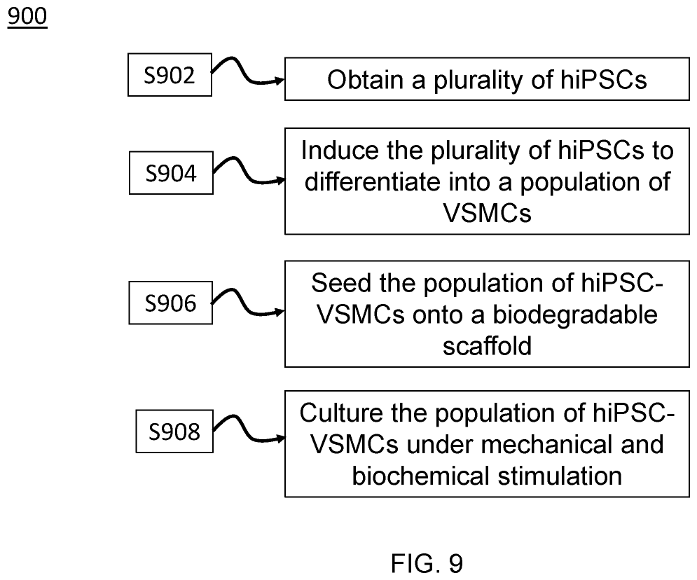

Referring to FIG. 9, embodiments of step S902 of method 900 include obtaining a plurality of hiPSCs. The hiPSCs may be obtained from a mammalian source including for example a human. The hiPSCs may be obtained by way of an allograft, an autograft and/or a xenograft. In some embodiments the hiPSCs are modulated to enhance their immunocompatibility. The hiPSCs with enhanced immunocompatibility may have modulated HLA expression. For example, the hiPSCs may have modulated expression of one or more of HLA-A alleles, HLA-B alleles, one or more class II HLAs, and/or combinations thereof. The immunocompatible hiPSCs may retain HLA-C expression.

Embodiments of step S904 include inducing the plurality of hiPSCs to differentiate into a population of hiPSC-VSMCs. The hiPSCs may be induced into hiPSC-VSMCs using differentiation media. The differentiation media may contain or omit one or more factors including for example TGF-β1. The differentiation media may contain one or more additional factors including serum, such as fetal bovine serum (FBS). The media may contain about 10% FBS. The media may contain between about 5% and about 15% FBS, about 1% to about 20% FBS, and/or increments thereof. The media may contain up to about 0.5% FBS, about 0.5% to about 1% FBS, about 1% to about 2% FBS, about 2% to about 3% FBS, about 3% to about 4% FBS, about 4% to about 5% FBS, about 5% to about 6% FBS, about 6% to about 7% FBS, about 7% to about 8% FBS, about 8% to about 9% FBS, about 9% to about 10% FBS or more than 10% FBS. The hiPSC-VSMCs may express one or more phenotypic markers of mature VSMCs including for example α-smooth muscle actin (α-SMA), calponin (CNN1), and smooth muscle myosin heavy chain (MYH11). The hiPSC-VSMCs may express one or more extracellular matrix (ECM) markers such as collagen type I (COL1) and elastin (ELN). In some embodiments, the plurality of hiPSCs are induced to differentiate into vascular endothelial cells (hiPSC-ECs).

Embodiments of step S906 include seeding the population of hiPSC-VSMCs onto a biodegradable scaffold. The biodegradable scaffold may include one or more biodegradable scaffolds as described herein. For example, the biodegradable scaffold may include a synthetic polymer scaffold such as a polyglycolic acid (PGA) scaffold. The biodegradable scaffold may include one or more of polyglycolic acid (PGA), polylactic acid (PLA), polycaprolactone, polyethylene glycol, polylactic-co-glycolic acid (PLGA), poly(glycerol sebacate) (PGS), fast-degrading polymers such as that comprising 87% glycolide, 7% trimethylene carbonate (TMC), and 6% polyethylene glycol, and/or combinations thereof. The biodegradable scaffold may be coated with one or more compounds including for example gelatin. The biodegradable scaffold may include one or more polymer meshes as described herein. The cells population of cells may be seeded onto the one or more polymer meshes at a density of up to about 20,000 cells/cm², about 20,000 cells/cm² to about 30,000 cells/cm², about 30,000 cells/cm² to about 40,000 cells/cm², about 40,000 cells/cm² to about 50,000 cells/cm² and the like.

Embodiments of step S908 of method 900 include culturing the population of hiPSC-VSMCs on the biodegradable scaffold under mechanical and biochemical stimulation for a duration of time, thereby generating a hiPSC-TEVG. The mechanical stimulation may include incremental radial stretching and pulsatile radial distension, as described herein. The scaffold seeded with cells may be mechanically loaded under pulsatile radial distension having a pulse rate of about 110 bpm to about 120 bpm, about 120 bpm to about 150 bpm, about 150 bpm to about 160 bpm, about 160 bpm to about 190 bpm, about 190 bpm to about 200 bpm, and all increments therebetween. The mechanical stimulation may include radial distention of up to about 0.5%, about 0.5% to about 1%, about 1% to about 1.5%, about 1.5% to about 2%, about 2% to about 2.5%, about 2.5% to about 3%, about 3% to about 3.5%, about 3.5% to about 4%, about 4% to about 4.5%, about 4.5% to about 5%, and increments therebetween. The scaffold seeded with cells may be mechanically stimulated using one or more techniques as understood in the art. For example, the scaffold seeded with cells may be attached to the outer surface of one or more lengths of distensible tubing. The tubing may then undergo pulsatile flow thereby applying pulsatile radial distention to the tubing and in turn the scaffold.

The biodegradable scaffold seeded with cells may undergo mechanical stimulation for a duration of up to 1 day, about 1 day to about 1 week, about 1 week to about 2 weeks, about 2 weeks to about 3 weeks, about 3 weeks to about 4 weeks, about 4 weeks to about 5 weeks, about 5 weeks to about 6 weeks, about 6 weeks to about 7 weeks, about 7 weeks to about 8 weeks, about 8 weeks to about 9 weeks, about 9 weeks to about 9 weeks, and increments therebetween.

The biodegradable scaffold seeded with cells may be cultured under biochemical stimulation including, for example TEVG culture media. The TEVG media may include one or more of DMEM medium supplemented with 20% (v/v) FBS, 50 μg/mL ascorbic acid (SIGMA-ALDRICH™), 50 μg/mL proline (SIGMA-ALDRICH™), 20 μg/mL alanine (SIGMA-ALDRICH™), 50 μg/mL glycine (SIGMA-ALDRICH™), 3 ng/mL CuSO₄ (SIGMA-ALDRICH™), 0.13 U/mL human insulin (SIGMA-ALDRICH™), 100 U/mL Penicillin G (SIGMA-ALDRICH™) and 1 ng/mL TGF-β1 (PEPROTECH™)). The TEVG media may additionally include or omit one or more of transforming growth factor-β1 (TGF-β1), platelet-derived growth factor-BB (PDGF-BB). The TEVG media may include TGF-β1 and PDGF-BB, TGF-β1 and not PDGF-BB, PDGF-BB and not TGF-β1, neither TGF-β1 nor PDGF-BB, and/or combinations thereof. The population of cells may be cultured under mechanical and biochemical stimulation for a duration up to and including 10 weeks.

In some embodiments, the TEVG is seeded with one or more populations of hiPSC-ECs and cultured using TEVG media and/or one or more variants thereof, thereby endothelializing the TEVG. The TEVG may be seeded with one or more populations of hiPSC-ECs, hiPSC-VSMCs, and or co-cultured with one or more combinations thereof. In some embodiments, the TEVG is cultured using or more populations of hiPSC-ECs, hiPSC-VSMCs, and or combinations thereof. The TEVG is cultured for a duration and then decellularized using one or more decellularization techniques as understood in the art and/or as described herein. The decellularized TEVG may retain one or more cell-derived factors including for example one or more cell-derived extracellular matrix constituents such as fibronectin, laminin, elastin, collagen (type I, II, III, IV, V, etc.), and the like. The TEVG may be reseeded with one or more populations of hiPSC-VSMCs, hiPSC-ECs, and/or combinations thereof.

Methods for Generating hiPSC-VSMCs

The hiPSC-VSMCs used may include hiPSC-VSMCs isolated from embryoid bodies using EDTA-mediated dissociation. The hiPSC-VSMCs used may include hiPSC-VSMCs isolated from embryoid bodies using EDTA-mediated dissociation and not dispase-associated dissociation. The isolated hiPSCs may be cultured in one or more transition media for a duration of time. The transition media may include one or more of mTeSR1 medium supplemented with 1:100 (v/v) GFR-MATRIGEL™ and 5 μM ROCK inhibitor (Y-27632; MILLIPORE™). The cells may be cultured in the transition media for a duration including up to about 4 days. The duration may include up to about 1 day, about 1 day to about 2 days, about 2 day to about 3 days, about 3 days to about 4 days, about 4 days to about 5 days, about 5 days to about 6 days, about 6 days to about 7 days, and the like. The cells may form embryoid bodies during this time. The media may then be transitioned from the transition media to an EB media for culturing the formed embryoid bodies. The EB media may include one or more of DMEM with high glucose (THERMOFISHER™) supplemented with 10% FBS, 2 mM L-glutamine, 1% (v/v) NEAA, 1% (v/v) pen/strep, and 0.012 mM β-ME. The transition media is transitioned to EB media by first diluting in 2:1 by volume on day 1, 1:1 by volume on day 2 and 1:2 by volume on day 3, respectively. The embryoid bodies may be cultured from day 4 to day 5 with EB medium in suspension. The embryoid bodies may then collected and seeded on a gelatin-coated culture dish for six days with EB medium. The embryoid bodies are then induced to transition to a VSMC lineage. The adherent EB-derived cells are dissociated by 0.05% trypsin-EDTA, re-seeded at 20,000 cells/cm² on GFR-MATRIGEL™-coated dishes, and cultured in SmGM-2 medium. The cells are cultured for a duration of time until they reach about 80% confluence. The duration of time may include about up to about 7 days, 7 days to 10 days, and so on. These proliferative, hiPSC-derived VSMCs cells at this stage are termed hiPSC-VSMCs-P. The hiPSC-VSMCs-P are then expanded for seeding onto the one or more vascular grafts as described herein. The hiPSC-VSMCs-P are passaged onto GFR-MATRIGEL™-coated plates or flasks and cultured in expansion medium (DMEM supplemented with 10% (v/v) FBS, 2 mM L-glutamine, 1% (v/v) NEAA, 1 mM sodium pyruvate and 1% (v/v) pen/strep) to “prime” the cells for an additional 1 or 2 passages. The hiPSC-VSMCs have effective proliferative potential. The hiPSC-VSMCs also express an enhanced level of one or more VSMC markers and one or more extracellular matrix proteins including, for example, collagen I and III. The process for deriving hiPSC-VSMCs for seeding onto TEVGs can take a period of time including about 4 weeks. The deriving of hiPSC-VSMCs may take about 2 weeks to about 3 weeks, about 3 weeks to about 4 weeks, about 4 weeks to about 5 weeks, about 5 weeks to about 6 weeks, about 6 weeks to about 7 weeks, about 7 weeks to about 8 weeks, or longer than about 8 weeks.

In some embodiments, the hiPSC-derived VSMCs are cultured in matrix-modifying culture media in order to modify the deposition of certain extracellular matrix proteins. The matrix-modifying media may contain one or more base constituents including DMEM medium containing 3% FBS and 1 ng/ml TGF-β1. The media may further contain one or more constituents that regulate the deposition of extracellular matrix proteins. The extracellular matrix protein may include, for example, elastin, collagen, and the like. That is, the media may be supplemented with one or more polyphenols including, for example, one or more of epigallocatechin gallate (EGCG), pentagalloyl (PGG), catechin, and/or one or more combinations thereof. The polyphenol may be supplemented into the media to a concentration of about 10 μg/ml. The polyphenol may be supplemented to the media to a concentration of up to about 0.5 μg/ml, from about 0.5 μg/ml to about 1 μg/ml, from about 1 μg/ml to about 2 μg/ml, from about 2 μg/ml to about 3 μg/ml, from about 3 μg/ml to about 4 μg/ml, from about 4 μg/ml to about 5 μg/ml, from about 5 μg/ml to about 6 μg/ml, from about 6 μg/ml to about 7 μg/ml, from about 7 μg/ml to about 8 μg/ml, from about 8 μg/ml to about 9 μg/ml, from about 9 μg/ml to about 10 μg/ml, from about 10 μg/ml to about 12 μg/ml, from about 12 μg/ml to about 14 μg/ml, from about 14 μg/ml to about 16 μg/ml, from about 16 μg/ml to about 18 μg/ml, from about 18 μg/ml to about 20 μg/ml, or greater than about 20 μg/ml including any and all increments therebetween. In some embodiments, the media is prepared with altered concentrations of one or more constituents that regulate the deposition of extracellular matrix deposition. For example, the media, may be prepared by modulating the concentration of one or more constituents such as PDGF-BB or TGF-β1 in order to enhance the collagen deposition while elastin deposition is enhanced by the addition of one or more constituents such as one or more polyphenol. The hiPSC-derived VSMCs may be cultured in the matrix-modifying culture media for a duration to sufficiently modify the deposition of one or more matrix proteins. For example, the hiPSC-derived VSMCs may be cultured in the matrix-modifying culture media for up to about 2 days, from about 2 days to about 4 days, for about 4 days to about 6 days, from about 6 days to about 8 days, from about 8 days to about 10 days, from about 10 days to about 12 days, from about 12 days to about 14 days, and/or longer than about 14 days including any and all increments therebetween. In some embodiments, the cells are cultured for a duration of about 9 days. In some embodiments, elevated ELN expression may be achieved by engineering doxycycline-inducible ELN into hiPSC-VSMCs. ELN deposition enhanced by polyphenol EGCG may be initiated before and/or during mechanical stimulation.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Introduction

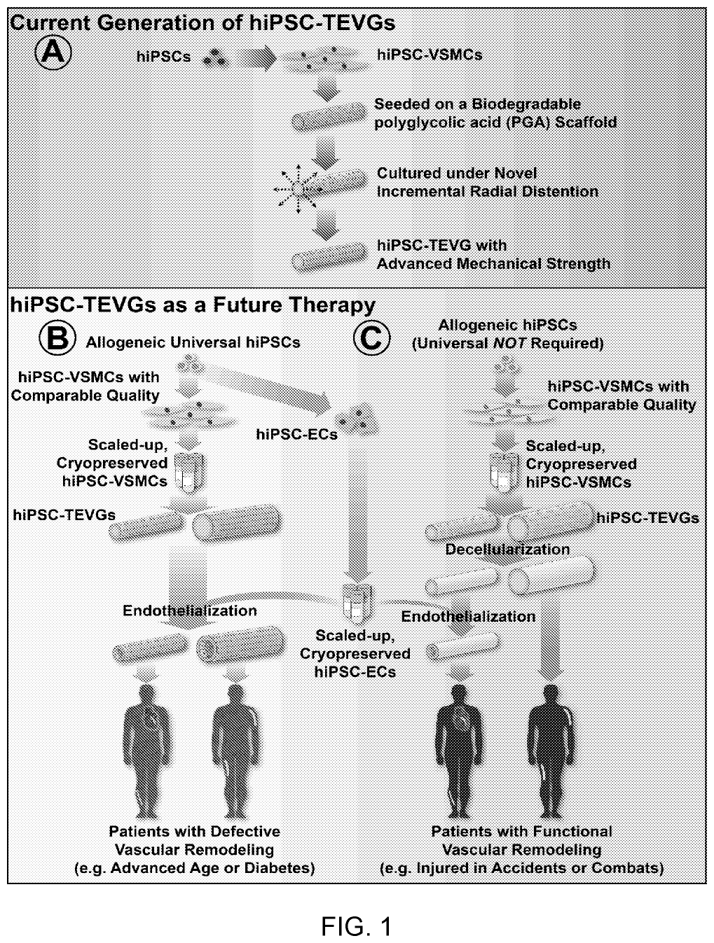

In the experiments described herein, hiPSC-VSMCs were cultured onto biodegradable polyglycolic acid (PGA) scaffolds, and developed hiPSC-TEVGs with remarkable mechanical strength approaching that of native vessels applied as typical arterial bypass grafts. The mechanical properties of TEVGs can be heightened by mechanical stretching to enhance collagen synthesis and proliferation of VSMCs (Kim et al., 1999, Nat Biotechnol 17, 979-983; Niklason et al., 1999, Science 284, 489-493). It was found that incremental addition of pulsatile radial stress at 110-120 beats per minute (bpm) significantly heightened the mechanical properties of the hiPSC-TEVGs (FIG. 1, Panel A). Moreover, after a four-week implantation as an interpositional aortic graft in a nude rat model, the hiPSC-TEVGs remained patent, without radial dilation, longitudinal elongation, or teratoma formation. The current, mechanically robust hiPSC-TEVGs provide a critical foundation for addressing patients with defective vascular remodeling, and pave the way for the production of “universal” hiPSC-TEVGs in the future as promptly available therapies (FIG. 1, Panel B). Additionally, for patients with functional vascular remodeling, functionally comparable hiPSC-VSMCs could be produced and stocked on a large scale, eliminating the donor-donor functional variability of primary cells, which could allow for more efficient production of decellularized hiPSC-TEVGs as an off-the-shelf therapy (FIG. 1, Panel C)

### Methods and Materials

### Wildtype Human Induced Pluripotent Stem Cells

The previously established wildtype human induced pluripotent stem cell (hiPSC) line Y6 was employed (Dash et al., 2016, Stem Cell Reports 7, 19-28; Gui et al., 2016, Biomaterials 102, 120-129). Y6 hiPSCs were originally produced through reprogramming fibroblasts derived from discarded female neonatal skin tissue using Sendai viral particles that encode human OCT4, KLF4, SOX2, and c-MYC genes (THERMOFISHER™). To maintain the pluripotency, hiPSCs were expanded in mTeSR1 medium (STEMCELL™ Technologies) on Growth Factor Reduced (GFR)-MATRIGEL™ (CORNING™)-coated plates under feeder-free conditions at 37° C. and were passaged every 5-7 days by Ethylenediaminetetraacetic acid (EDTA; THERMOFISHER™) treatment.

### HLA-C-Retained (CIITA-Knockout) Human Induced Pluripotent Stem Cells

The human leukocyte antigen-C(HLA-C) retained (CIITA-knockout) hiPSC line (named as 585A1 hiPSCs in previously published study) was established via reprogramming peripheral blood mononuclear cells isolated from male donor by episomal vectors as previously described (Xu et al., 2019, Cell Stem Cell 24, 566-578 e567). These HLA-C-retained hiPSCs were expanded in mTeSR1 medium on GFR-MATRIGEL™-coated plates under feeder-free conditions at 37° C. and were passaged every 5-7 days by EDTA treatment.

### Human Vascular Smooth Muscle Cells

Primary human vascular smooth muscle cells (VSMCs) derived from the aorta of male donors were purchased from LONZA™. The primary human VSMCs were expanded in Smooth Muscle Growth Medium (SmGM-2; Lonza) on 0.1% (w/v) gelatin (SIGMA-ALDRICH™)-treated culture dishes at 37° C. and were passaged upon reaching 80% confluency by 0.05% trypsin-EDTA (THERMOFISHER™) treatment. To further induce the maturation phenotype of primary VSMCs for characterization of marker expression and contractile functions, primary VSMCs grown in SmGM-2 medium were subcultured in the VSMC maturation medium (Dulbecco's Modified Eagle Medium (DMEM; high glucose, THERMOFISHER™) with 1% (v/v) fetal bovine serum (Fetal Bovine Serum, FBS; GEMINI™), 2 mM L-glutamine (THERMOFISHER™), 1% (v/v) non-essential amino acid (NEAA; THERMOFISHER™), 1% (v/v) penicillin/streptomycin (pen/strep; THERMOFISHER™), 0.012 mM 2-mercaptoethanol (β-ME; THERMOFISHER™) and 1 ng/ml TGFβ1 (PEPROTECH™)) for seven days.

### Human Umbilical Vein Endothelial Cells

Human umbilical vein endothelial cells (HUVECs) derived from the umbilical cords of female donors were purchased from Lonza. The HUVECs were expanded in Endothelial Growth Medium (EGM-2; LONZA™) on gelatin-treated culture dishes at 37° C. and were passaged upon reaching 80% confluency by 0.05% trypsin-EDTA treatment.

### Animal Use

NIH-Foxn1rnu nude rats were obtained from Charles River Laboratories. All experiments were performed on male nude rats 10 weeks of age, weighing about 300 g.

### Human Umbilical Arteries

Human umbilical cords (deidentified) were obtained from Yale-New Haven Hospital (New Haven, Conn.), delivered at 4° C., and processed immediately after delivery. Human umbilical arteries (HUAs) were then isolated from the umbilical cords (20-30 cm in length) within 30 minutes via sharp dissection in a sterile manner. A pair of Metzenbaum scissors were used to remove the Wharton's jelly surrounding the HUAs. The newly isolated HUAs were then gently washed with Dulbecco's Phosphate-Buffered Saline (DPBS; THERMOFISHER™) containing penicillin 100 U/mL and streptomycin 100 μg/mL (THERMOFISHER™) to remove blood clots and were immediately used for either biomechanical analysis or were subjected to histological analysis.

### Porcine Coronary Arteries

The porcine coronary arteries were isolated from Yorkshire pigs (male, three-month-old) in 20 minutes after euthanization via Veterinary Clinical Services from Yale Animal Resources Center. Coronary arteries were immediately transferred to DPBS containing penicillin 100 U/mL and streptomycin 100 μg/mL (THERMOFISHER™) at 4° C. The adherent connective tissue and fat tissue were removed in a sterile manner, and the segments of arteries with inner diameters of approximately 3 mm were cut into vessel rings with 1-2 mm in length for evaluation of mechanical strength. The vessel rings from coronary arteries of three Yorkshire pigs were used to mechanical strength evaluation.

Generation of VSMCs from hiPSCs

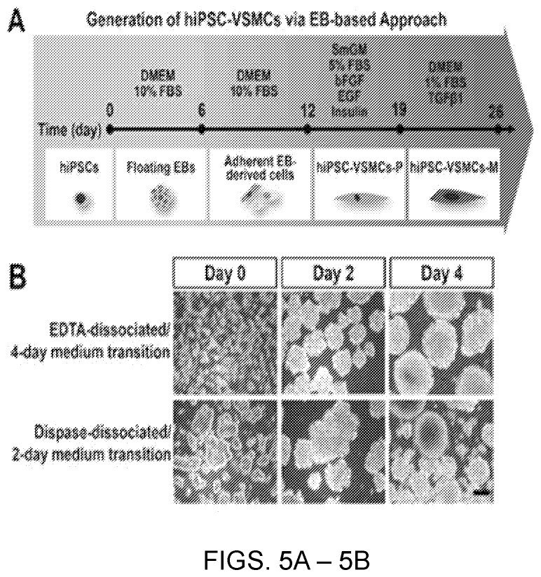

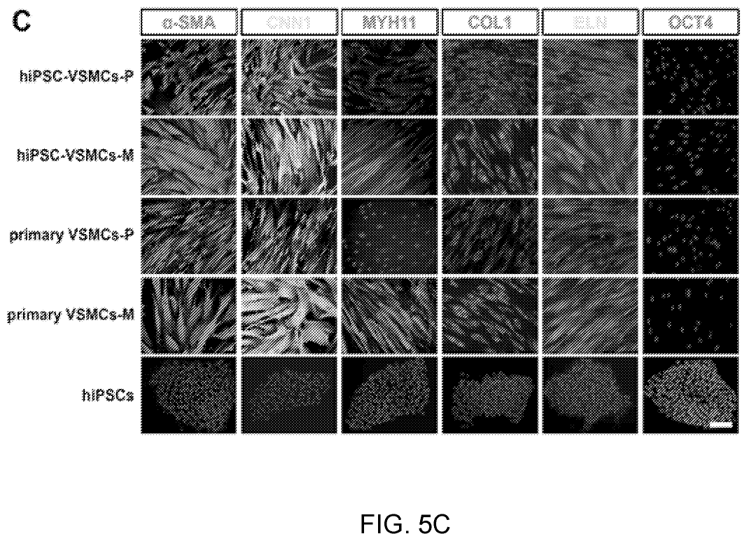

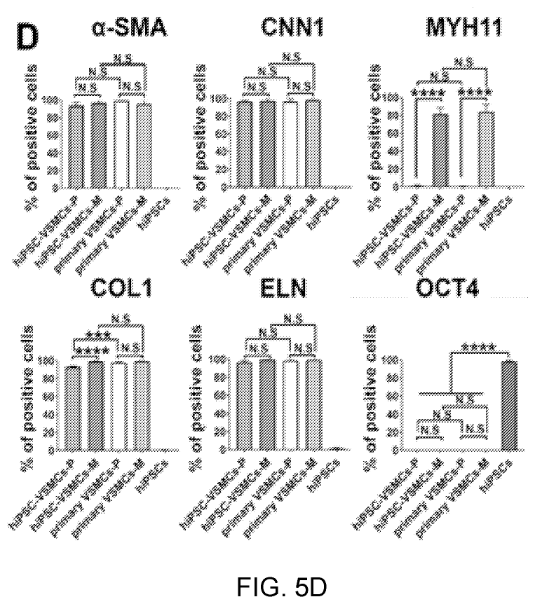

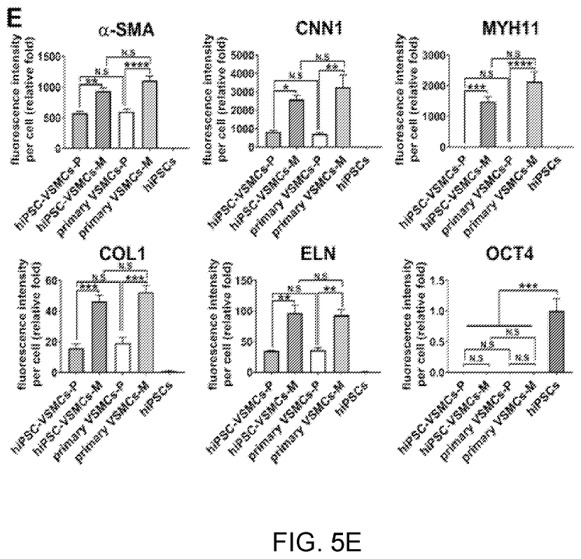

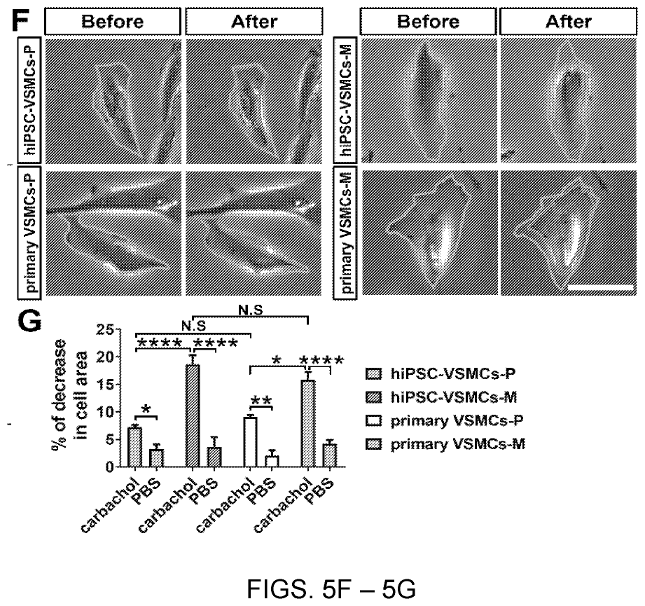

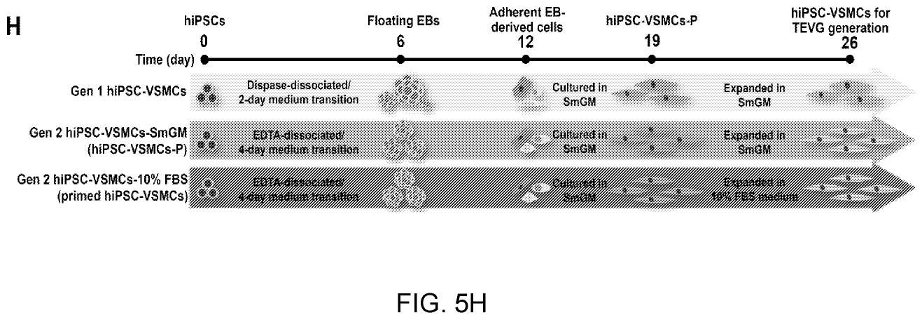

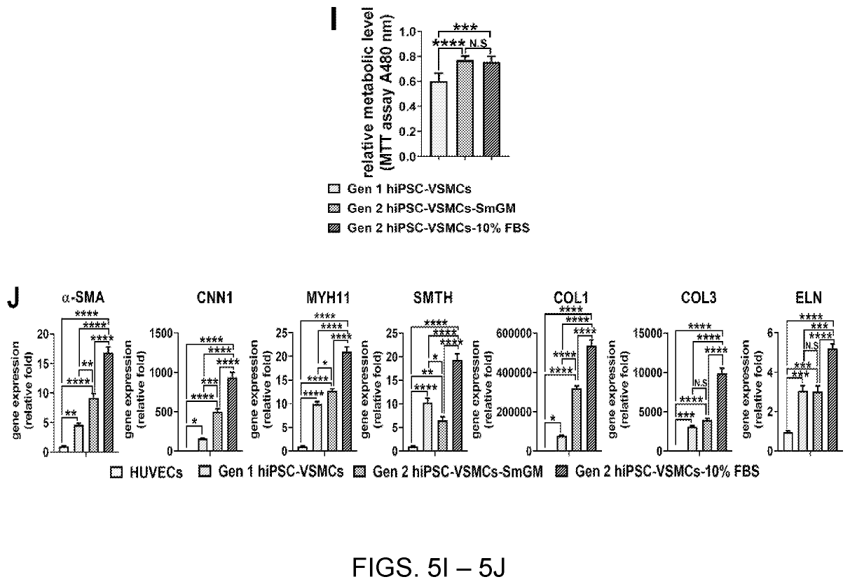

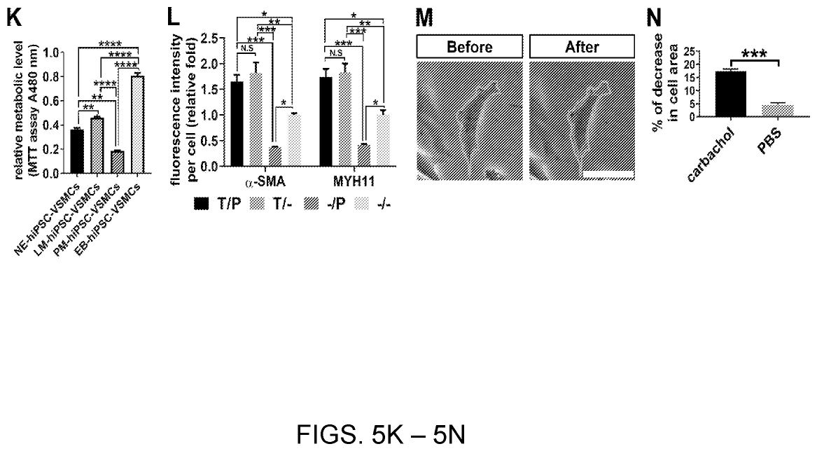

The hiPSC-VSMCs were obtained via an embryoid body (EB)-based approach (Dash et al., 2016, Stem Cell Reports 7, 19-28; Gui et al., 2016, Biomaterials 102, 120-129), with significant modifications (FIG. 5A). Briefly, hiPSCs were expanded until 80% confluency and treated with 0.5 mM EDTA (THERMOFISHER™) for 3 minutes at 37° C. on Day 0. The dissociated cells (3 wells of a 6-well plate) were resuspended in mTeSR1 medium supplemented with 1:100 (v/v) GFR-MATRIGEL™ and 5 μM ROCK inhibitor (Y-27632; MILLIPORE™) and transferred to a 6-well low attachment plate (3 wells; CORNING™) for 24 hours, which allowed the formation of EBs with uniform size. The mTeSR1 medium (hiPSC self-renewal medium) was gradually mixed with EB medium (DMEM high glucose (THERMOFISHER™) supplemented with 10% FBS, 2 mM L-glutamine, 1% (v/v) NEAA, 1% (v/v) pen/strep, and 0.012 mM (3-ME) in 2:1 (volume) on Day 1, 1:1 on Day 2 and 1:2 on Day 3, respectively. From Day 4 to Day 5, EBs were cultured with EB medium in suspension. EBs were then collected and seeded on a gelatin-coated culture dish for six days with EB medium. To induce VSMC lineage, the adherent EB-derived cells were dissociated by 0.05% trypsin-EDTA, re-seeded at 20,000 cells/cm2 on a GFR-MATRIGEL™-coated dish, and cultured in SmGM-2 medium until cells reached 80% confluence. This stage typically took 7-10 days, and these proliferative hiPSC-derived VSMCs were termed hiPSC-VSMCs-P. In order to expand hiPSC-VSMCs-P for vascular graft engineering, hiPSC-VSMCs-P were passaged to GFR-MATRIGEL™-coated plates or flasks and cultured in expansion medium (DMEM supplemented with 10% (v/v) FBS, 2 mM L-glutamine, 1% (v/v) NEAA, 1 mM sodium pyruvate (THERMOFISHER™) and 1% (v/v) pen/strep) to “prime” the cells for an additional 1 or 2 passages. This led to the generation of hiPSC-VSMCs with effective proliferative potential while also expressing an enhanced level of VSMC markers and extracellular matrix proteins collagen I and III. The derivation of hiPSC-VSMCs for tissue engineered vascular graft (TEVG) generation typically took around four weeks.

To further induce the maturation phenotype of hiPSC-VSMCs (hiPSC-VSMCs-M) for characterization of marker expression and contractile functions, hiPSC-VSMCs-P grown in SmGM-2 medium were subcultured in the VSMC maturation medium (DMEM with 1% FBS, 1% (v/v) NEAA, 2 mM L-glutamine, 0.012 mM β-ME and 1 ng/ml TGF-β1) for seven days.

The lineage specific hiPSC-VSMCs (including hiPSC-VSMCs with embryonic origin of neuroectoderm, lateral plate mesoderm, or paraxial mesoderm) were derived following the chemically-defined method as previously reported (Cheung et al., 2012, Nat Biotechnol 30, 165-173). The lineage specific hiPSC-VSMCs were subcultured in expansion medium (DMEM supplemented with 10% (v/v) FBS, 2 mM L-glutamine, 1% (v/v) NEAA, 1 mM sodium pyruvate and 1% (v/v) pen/strep) along with those hiPSC-VSMCs derived from the newly optimized EB-based method for comparison of proliferative capacity.

### Immunostaining of Cultured Cells

Cells were washed with DPBS and fixed in 4% paraformaldehyde (PFA; Electron Microscopy Sciences) for 10 minutes at room temperature (RT). The cells were then blocked in 10% normal goat serum (NGS; THERMOFISHER™) in PBST buffer (DPBS with 0.3% Triton X-100 (SIGMA-ALDRICH™)) for 30 minutes at RT. Subsequently, cells were incubated with the primary antibody in 1% NGS in PBST at 4° C. overnight. Cells were washed again with DPBS, incubated with secondary antibody (1:1000 in 1% NGS in PBST) for one hour at RT and washed with DPBS again. Filamentous actin was stained with phalloidin (THERMOFISHER™) and the nuclei were counterstained with DAPI (THERMOFISHER™). All antibodies are listed in the Table 1. Immunostained samples were analyzed using a fluorescent microscope (LEICA™ Microsystems). Fluorescence intensity (gray scale) of the markers in immunostained cells were analyzed with the ImageJ software and expressed relatively to cell number. Percentage of cells positive for the markers was quantified by using ImageJ software.

TABLE 1
Key Resources

| | | |
|---|---|---|
| REAGENT or RESOURCE | SOURCE | IDENTIFIER |

Antibodies

| | | |
|---|---|---|
| α-SMA | SIGMA ™ | A5228 |
| CNN1 | SIGMA ™ | C2687 |
| MYHII | ABCAM ™ | ab53219 |
| COLI | ABCAM ™ | ab34710 |
| ELN | ABCAM ™ | ab21610 |
| OCT4 | ABCAM ™ | ab18976 |
| N-cadherin | SANTA CRUZ | sc-7939 |
| | BIOTECHNOLOGY ™ | |
| HLA-A | ABCAM ™ | ab52922 |
| HLA-A | SANTA CRUZ | sc365485 |
| | BIOTECHNOLOGY ™ | |
| KI67 | BD BIOSCIENCE ™ | 550609 |
| CD31 | ABCAM ™ | ab28364 |
| CD68 | ABCAM ™ | ab31630 |
| vWF | SANTA CRUZ | sc-8068 |
| | BIOTECHNOLOGY ™ | |
| Alexa 488 goat anti-mouse IgG | THERMOFISHER ™ | A11029 |
| Alexa 488 goat anti-rabbit IgG | THERMOFISHER ™ | A11008 |
| Alexa 555 goat anti-mouse IgG | THERMOFISHER ™ | A21426 |
| Alexa 555 goat anti-rabbit IgG | THERMOFISHER ™ | A21428 |
| Alexa 647 goat anti-mouse IgG | THERMOFISHER ™ | A21235 |
| Alexa 647 goat anti-rabbit IgG | THERMOFISHER ™ | A21245 |
| Alexa 488 donkey anti-goat IgG | THERMOFISHER ™ | A11055 |

Biological Samples

| | | |
|---|---|---|
| human umbilical cord arteries | Yale-New Haven Hospital | N/A |
| porcine coronary arteries | Yale University, Veterinary Clinical | N/A |
| | Services | |

Chemicals, Peptides, and Recombinant Proteins

| mTeSR ™1 medium kit | STEMCELL ™ Technology | 85850 |
|---|---|---|
| Ethylenediaminetetraacetic acid (EDTA) | THERMOFISHER ™ | 15575020 |
| Dispase II | THERMOFISHER ™ | 17105041 |
| MATRIGEL ™, growth factor reduced | BD BIOSCIENCSES ™ | 354230 |
| | CORNING ™ | |
| Y-27632 | MILLIPORE ™ | 1254 |
| Dulbecco's Modified Eagle Medium | THERMOFISHER ™ | 11965-092 |
| Fetal bovine serum | GEMINI ™ | 100-106 |
| L-glutamine | THERMOFISHER ™ | 25030-081 |
| Non-essential amino acid | THERMOFISHER ™ | 11140-050 |
| Penicillin/streptomycin | THERMOFISHER ™ | 15140-122 |
| β-mercaptoethanol | SIGMA-ALDRICH ™ | M3148-25 ml |
| 0.05% trypsin-EDTA | THERMOFISHER ™ | 25300-054 |
| Sodium pyruvate | THERMOFISHER ™ | 11360-070 |
| Recombinant human TGFPl | PEPROTECH ™ | 100-21C-10UG |
| Recombinant human PDGF-BB | PEPROTECH ™ | 220-BB-050 |
| Ham's F-12 nutrient mix | THERMOFISHER ™ | 11765054 |
| Iscove's Modified Dulbecco's Medium | THERMOFISHER ™ | 12440053 |
| (IMDM) | | |
| Chemically-defined lipid concentrate | THERMOFISHER ™ | 11905031 |
| Monothioglycerol | SIGMA-ALDRICH ™ | M6145 |
| Transferrin | ROCHE ™ | 652202 |
| Insulin (for lineage-specific VSMC | ROCHE ™ | 1376497 |
| differentiation) | | |
| Poly(vinyl alcohol) | SIGMA-ALDRICH ™ | P8136 |
| Recombinant human BMP4 | R&D SYSTEMS ™ | 314-BP |
| Recombinant human FGF2 | R&D SYSTEMS ™ | 233-FB |
| LY294002 | SIGMA-ALDRICH ™ | L9908 |
| SB431542 | SIGMA-ALDRICH ™ | S4317 |
| Gelatin | THERMOFISHER ™ | G2500-100G |
| Smooth Muscle Growth Medium-2 | LONZA ™ | CC-3182 |
| (SmGM) Bullet Kit | | |
| Endothelial Cell Growth Medium-2 (EGM- | LONZA ™ | CC-3162 |
| 2) Bullet Kit | | |
| Normal goat serum | THERMOFISHER ™ | 10000C |
| Dulbecco's Phosphate-Buffered Saline | THERMOFISHER ™ | 14190-144 |
| Triton X-100 | AmericanBIO | AB02025-00500 |
| 4,6-diamidino-2-phenylindole (DAPI) | THERMOFISHER ™ | D1306 |
| Alexa Fluor ™ 568 Phalloidin | THERMOFISHER ™ | A12380 |
| Carbachol | ABCAM ™ | abl41354 |
| 3-(4,5 -dimethylthiazol-2-yl)-2,5-diphenyl | SIGMA-ALDRICH ™ | M2128 |
| tetrazolium bromide (MTT) | | |
| Nonwoven-PGA polymer mesh, 1.0 mmx5- | BIOFELT ™ | N/A |
| 6 mg/cm2, 20 cm × 30 cm | | |
| Sodium hydroxide | SIGMA-ALDRICH ™ | S0899 |
| Ascorbic acid | SIGMA-ALDRICH ™ | A4544-25 g |
| Proline | SIGMA-ALDRICH ™ | P5607-25 g |
| Glycine | SIGMA-ALDRICH ™ | G8790-100 g |
| Alanine | SIGMA-ALDRICH ™ | A7469-25 g |
| CuSO₄ | SIGMA-ALDRICH ™ | C8027-500G |
| Penicillin G | SIGMA-ALDRICH ™ | |
| Human recombinant insulin (for TEVG | SIGMA-ALDRICH ™ | 19080-50 mg |
| culture) | | |
| Sodium chloride | SIGMA-ALDRICH ™ | S9625 |
| Potassium chloride | SIGMA-ALDRICH ™ | P5405 |
| Magnesium chloride | SIGMA-ALDRICH ™ | M8266 |
| HEPES | SIGMA-ALDRICH ™ | H3375 |
| Dextrose | SIGMA-ALDRICH ™ | D9434 |
| Calcium chloride dihydrate | SIGMA-ALDRICH ™ | C7902 |
| Sucrose | SIGMA-ALDRICH ™ | S3089-1 kg |
| Tissue-Tek O.C.T. Compound | Sakura Finetek | 4583 |
| Ultra-pure agarose | SIGMA-ALDRICH ™ | 16500-500 |

Critical Commercial Assays

| TRIzol ™ RNA Isolation Kit | THERMOFISHER ™ | |
|---|---|---|
| iScript cDNA synthesis Kit | BIO-RAD ™ | 170-8891 |
| iQ SYBR ™ Green Supermix | BIO-RAD ™ | 1708882 |
| Hydroxyproline assay kit | SIGMA-ALDRICH ™ | MAK008 |
| TUNEL staining kit | ROCHE ™ | 11684795910 |
| ATP Assay Kit (Colorimetric) | ABCAM ™ | ab83355 |

Experimental Models: Cell Lines

| | | |
|---|---|---|
| Y6 human induced pluripotent stem cells | Yale University, Stem Cell Research | N/A |
| | Center | |
| HLA-C retained (CIITA) human induced | Center for iPS Cell Research and | N/A |
| pluripotent stem cells (585Al-C7-only#3- | Application (CiRA), Kyoto | |
| 1 + CIITA-ex3g5-NF #6) | University | |
| Primary human aortic smooth muscle cells | LONZA ™ | cc-2571 |
| Human umbilical vein endothelial cells | LONZA ™ | cc-2517 |

Experimental Models: Organisms/Strains

| | | |
|---|---|---|
| NIH-Foxnlrnu nude rats (10-week-old, | Charles River | NIH-Foxn1rnu |
| male, 300 g) | | nude rats |

Oligonucleotides

| | | |
|---|---|---|
| qRT-PCR primers | herein | Table 2 |

Software and Algorithms

| | | |
|---|---|---|
| ImageJ | National Institutes of Health | https://imagej.nih.gov/ij/ |
| GraphPad Prism 8 | GraphPad Software | https: //www.graphpad.com/ |

Other

| | | |
|---|---|---|
| Costar ® Ultra-Low Attachment 6 Well | CORNING™ | 3471 |
| Plate | | |
| Costar ® Ultra-Low Attachment 24 Well | CORNING™ | 3473 |
| Plate | | |
| UniFlex ® Culture Plates | FLEXCELL ™ International | UF-4001U-Each |
| | Corporation | |
| FX-6000T ™ Tension System | FLEXCELL ™ International | N/A |
| | Corporation | |
| Glass bioreactors for TEVG culture | Yale University, Scientific | N/A |
| | Glassblowing Laboratory | |
| COLE-PARMER ™ PTFE Syringe Filters, | COLE-PARMER ™ | EW-02915-08 |
| Sterile; 0.20 μm, 25 mm Diameter (Air | | |
| filter of TEVG bioreactor) | | |
| MASTERFLEX ™ Replacement controller | COLE-PARMER ™ | EW-07553-71 |
| for 07553-series L/S systems, 115V | | |
| (peristaltic pump for TEVG culture) | | |
| MASTERFLEX ™ L/S Easy-Load ® II | COLE-PARMER ™ | EW-77200-60 |
| Head for Precision Tubing, PPS/SS (Head | | |
| of peristaltic pump) | | |
| MASTERFLEX ™ L/S PharMed BPT | COLE-PARMER ™ | EW-06508-18 |
| Tubing, L/S #18, 25 ft (connection tubing | | |
| for bioreactor, large) | | |
| MASTERFLEX ™ L/S BioPharm Platinum- | COLE-PARMER ™ | EW-96420-16 |
| Cured Silicone Pump Tubing, L/S 16 | | |
| (connection tubing for bioreactor, small) | | |
| MASTERFLEX ™ L/S PharMed BPT | COLE-PARMER ™ | EW-06508-16 |
| Tubing, L/S #16 (feeding tubing) | | |
| 4-0 SURGIPRO ™ blue 36″ cv-25 taper, | MEDTRONIC ™ Animal Health | VP761X |
| double armed (surgical suture to stitch PGA | | |
| scaffold to the Dacron arm) | | |
| Coated Vicryl ® (polyglactin 910) suture | ETHICON ™ | J492G |
| (surgical suture to sew the tubule PGA | | |
| scaffold around the silicon tube) | | |
| HEMASHIELD ™ Platinum Woven Double | MAQUET ™ | 175438P |
| Velour Vascular Grafts (Dacron to anchor | | |
| the PGA scaffold to the glass arm) | | |
| Silicon tube (inner diameter 3.2 mm) | SAINT-GOBAIN ™ | F05027 |
| EDWARDS ™ Truwave DPT Px600f | EDWARDS ™ Lifesciences | PX212 |
| Pressure Monitoring Set 12″ | | |
| 4-way stopcock | EDWARDS ™ Lifesciences | 594WSC |
| Injection Site Interlink ® | MCKESSON ™ Medical | 2N3399 |
| 6-0 ETHIBOND ™ green 1 × 24″ rb-4 double | ETHICON ™ | D9591 |
| armed (surgical suture for suture retention | | |
| test) | | |
| GlucCell Glucose Monitoring System | CESCO Bioengineering | DG1000 |
| Glucose Test Strips | CESCO Bioengineering | DGA050 |
| INSTRON ™ 5960 mechanical testing | INSTRON ™ | N/A |
| system | | |
| SI-H KG7 Force Transducers | World Precision Instruments | N/A |

### Quantitative Reverse Transcription PCR

Cells or engineered vascular tissues were subjected to RNA extraction and a quantitative reverse transcription PCR (qRT-PCR) assay to evaluate the gene expression of markers of interest. RNA extraction and purification were completed using the TRIzol™ RNA Isolation Kit (THERMOFISHER™), following the manufacturer's instructions. Subsequently, total RNA was subjected to reverse transcription using an iSCRIPT™ cDNA synthesis Kit (BIO-RAD™). The primer sequences of the genes used in qRT-PCR are listed in Table 2. qRT-PCR was performed using BIO-RAD™ IQ SYBR® green supermix. Expression of genes of interest was normalized to that of human GAPDH. Three biological replicates were used for the analysis of each gene expression.

TABLE 2
List of Primers for qRT-PCR

| | | SEQ |
|---|---|---|
| | | ID |
| Gene | Primer sequence | NO |
| GAPDH-F | TGTTGCCATCAATGACCCC | 1 |
| | TT | |
| GAPDH-R | CTCCACGACGTACTCAGCG | 2 |
| α-SMA-F | CTGGGACGACATGGAAAA | 3 |
| α-SMA-R | ACATGGCTGGGACATTGA | 4 |
| CNN1-F | AGCATGGCGAAGACGAAA | 5 |
| | GGAA | |
| CNN1-R | CCCATCTGCAGGCTGACA | 6 |
| | TTGA | |
| MYH11-F | AGAGACAGCTTCACGAGTAT | 7 |
| | GAG | |
| MYH11-R | CTTCCAGCTCTCTTTGAAA | 8 |
| | GTC | |
| SMTH-F | CCTGGATACAGAGGACATGG | 9 |
| SMTH-R | CAGGTGGTTGTAGAGCGACT | 10 |
| COL1-F | CCTGTCTGCTTCCTGTAAA | 11 |
| | CTC | |
| COL1-R | GTTCAGTTTGGGTTGCTTG | 12 |
| | TC | |
| COL3-F | GCTCTGCTTCATCCCACTA | 13 |
| | TTA | |
| COL3-R | CTGGCTTCCAGACATCTCT | 14 |
| | ATC | |
| ELN-F | AAGATGGTGCAGACACTTCC | 15 |
| ELN-R | AGAGCGAATCCAGCTTTGAG | 16 |
| vinculin-F | AATGGTCCAGCAAGGGCAAT | 17 |
| vinculin-R | GAATGAGTGCCCGCTTGGTA | 18 |
| N-cadherin- | GGGAAATGGAAACTTGATGG | 19 |
| F | CA | |
| N-cadherin- | CAGTTGCTAAACTTCACTGA | 20 |
| R | AAGGA | |
| GLUT1-F | TGGCATCAACGCTGTCTTCT | 21 |
| GLUT1-R | AGCCAATGGTGGCATACACA | 22 |
| GLUT4-F | GGTTCTTTCATCTTCGCCGC | 23 |
| GLUT4-R | TCCCCATCTTCGGAGCCTAT | 24 |
| CS-F | CTCAACTCAGGACGGGTTGT | 25 |
| CS-R | GGGGTCATTAGGCAGGTGTT | 26 |
| PGC1α-F | CTACGGCTCCTCCTGGGAAA | 27 |
| PGC1α-R | CAGTCCAGGGGCAGAAAAGT | 28 |

### Cell Contractility Assay

hiPSC-VSMCs or human primary VSMCs cultured in either SmGM-2 medium, maturation medium (1% (v/v) FBS with 1 ng/ml TGF-β1 (Peprotech)), or TEVG medium (DMEM medium supplemented with 20% (v/v) FBS, 50 μg/mL ascorbic acid (SIGMA-ALDRICH™), 50 μg/mL proline (Sigma-Aldrich), 20 μg/mL alanine (SIGMA-ALDRICH™), 50 μg/mL glycine (SIGMA-ALDRICH™), 3 ng/mL CuSO₄(SIGMA-ALDRICH™), 0.13 U/mL human insulin (SIGMA-ALDRICH™), 100 U/mL Penicillin G (SIGMA-ALDRICH™) and 1 ng/mL TGF-β1 (Peprotech)) were treated with 1 mM carbachol (ABCAM™) or DPBS (vehicle control) as a control for 20 minutes. Cell surface areas were recorded at the beginning and the end of the treatment. The changes of surface area were evaluated with the ImageJ software. Three independent batches of hiPSC-VSMCs or primary VSMCs were used in the contractility assay, and the changes in surface area of 10 randomly selected cells in each batch were recorded and analyzed, respectively.

### MTT Assay

hiPSC-VSMCs derived from EB-based or chemically defined approach were seeded at 20,000 cells/well density into GFR-MATRIGEL™-coated 96-well plates with expansion medium and cultured for three days. Cell proliferation was measured as a function of metabolic activity using 3-(4, 5-dimethylthiazol-2-yl)-2, 5-diphenyl tetrazolium bromide (MTT) (SIGMA-ALDRICH™) on Day 4. MTT at 0.5 mg/ml was added into the medium of each well and incubated with the cells at 37° C. for 2 hours, followed by cell solubilization with DMSO (AmericanBIO) for 15 minutes. Absorbance was measured at 540 nm using the Synergy 2 multi-mode plate reader (BIOTEK™). Three biological replicates were completed for evaluation of each cell group.

Culturing hiPSC-VSMCs on 5 mm×5 mm PGA Scaffolds

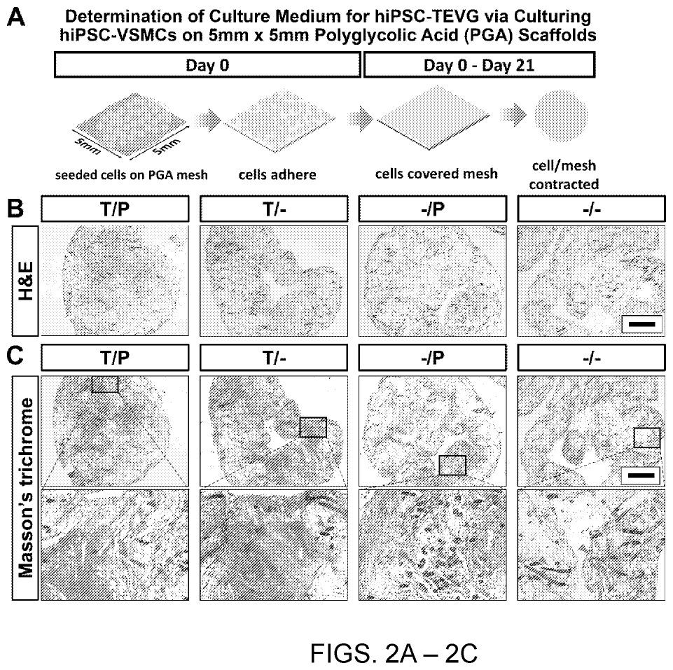

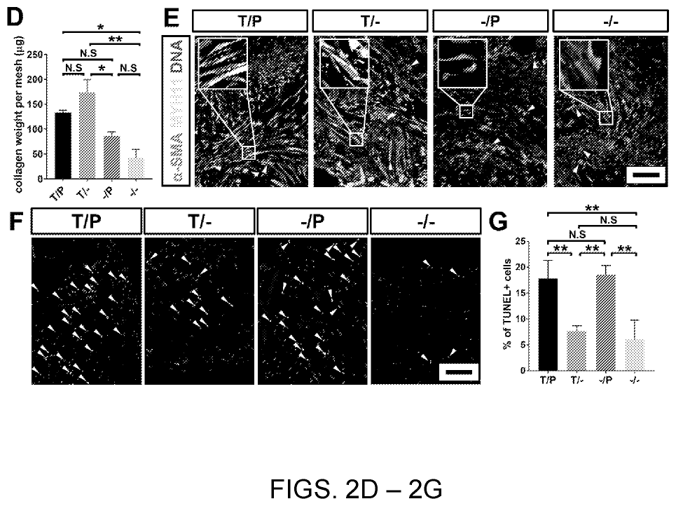

The engineered vascular tissues were developed by seeding “primed” hiPSC-VSMCs onto PGA scaffolds, to evaluate the effect of growth factor components (TGF-β1 and PDGF-BB) in the TEVG medium (FIG. 2A). Nonwoven-PGA polymer mesh (0.3 mm×150 mg/cc, 20 cm×30 cm sheet, BIOFELT™) was cut into small-size meshes (5 mm×5 mm squares). The PGA squares were treated with 1.0 N NaOH (SIGMA-ALDRICH™) for 1 minute, rinsed extensively with distilled water, sterilized with 70% ethanol, and air-dried overnight in a sterile manner. On the following day, PGA meshes were coated with 0.1% gelatin at 37° C. for 1 hour, air-dried, and transferred into the 24-well low attachment dish (CORNING™).

“Primed” hiPSC-VSMCs cultured in the expansion medium (DMEM supplemented with 10% (v/v) FBS, 2 mM L-glutamine, 1% (v/v) NEAA, 1 mM sodium pyruvate (THERMOFISHER™) and 1% (v/v) pen/strep) were harvested, and 40 μL of the expansion medium containing 0.4 million hiPSC-VSMCs were dropped onto the PGA mesh and incubated at 37° C. and 5% CO₂ for one hour. The wells were then filled with 1 mL of expansion medium, and cells were cultured overnight at 37° C. On the following day, the medium was changed to the TEVG medium (DMEM medium supplemented with 20% (v/v) FBS, 50 μg/mL ascorbic acid (SIGMA-ALDRICH™), 50 μg/mL proline (SIGMA-ALDRICH™), 20 μg/mL alanine (SIGMA-ALDRICH™), 50 μg/mL glycine (SIGMA-ALDRICH™), 3 ng/mL CuSO₄ (SIGMA-ALDRICH™), 0.13 U/ml human insulin (SIGMA-ALDRICH™) and 100 U/ml Penicillin G (SIGMA-ALDRICH™)), supplemented with one of the following: 1) both human TGF-β1 (1 ng/ml, PEPROTECH™) and human PDGF-BB (10 ng/ml, R&D SYSTEMS™) (T/P), 2) TGF-β1 only (T/−), 3) PDGF-BB only (−/P), or 4) no growth factor (−/−). Medium was changed every other day. The tissues were cultured for 21 days and then harvested for both histological analysis and hydroxyproline assay.

### Tissue Immunohistochemistry and Histology

Tissue samples were fixed in 4% PFA for three hours at RT and incubated in 15% (w/v) sucrose (SIGMA-ALDRICH™) in DPBS at 4° C. for 16 hours. Subsequently, the fixed tissues were embedded in Tissue-Tek Optimal Cutting Temperature (OCT) Compound (Sakura Finetek) to develop the frozen blocks. Frozen blocks were sectioned at 5 μm intervals using cryostat (LEICA™ CM1950) and the sections were stored at −80° C.

For immunostaining, slides were submerged in PBS for 10 minutes. Tissue sections were then incubated with PBST containing 10% NGS for 30 minutes at RT, and incubated with primary antibody in PBST containing 1% NGS at 4° C. overnight in a humidified chamber. On the second day, sections were washed with DPBS, incubated with secondary antibody (1:1000 in PBST with 1% NGS) for one hour at RT and washed with DPBS again. Nuclei were counterstained with DAPI. Fluorescence intensity (gray scale) of positiveness of the markers in the total immunostained cells or HLA-A-positive cells were analyzed by using ImageJ software and expressed relatively to cell number. Percentage of positiveness of the markers in the HLA-A-positive cells were analyzed by using ImageJ software.

For hematoxylin and eosin (H&E) staining, Masson's Trichrome staining, Alizarin Red staining and elastic Verhoeff-Van Gieson staining (EVG staining), tissue sections were processed based on standard protocols.

### TUNEL Assay

Tissue sections were processed according to the instructions from the TUNEL staining kit (ROCHE™). DNA was counterstained by DAPI (THERMOFISHER™). Three biological replicates were completed for each group for statistical analysis of the percentage of TUNEL-positive cells. All immunofluorescence, histology and TUNEL staining micrographs were captured under an inverted microscope (Nikon Eclipse 80i).

### Hydroxyproline Assay

The collagen weight of the vascular tissues or hiPSC-VSMCs cultured in UNIFLEX™ 6-well culture plates (FLEXCELL™ International Corporation) were determined by measuring the levels of hydroxyproline following the instruction of the hydroxyproline assay kit (SIGMA-ALDRICH™). Collagen weight was then calculated based on the estimation that collagen contains approximately 10.0% hydroxyproline by weight (Dijkman et al., 2012, Biomaterials 33, 4545-4554; Piez and Likins, 1960, The nature of collagen. In Calcification in biological systems A symposium presented at the Washington meeting of the American Association for the Advancement of Science, Dec. 29, 1958, R. F. Sognnaes, ed. (American Association for the Advancement of Science), pp. 411-420). Three biological replicates were completed for each group.

Cell Culture with Pulsatile Uniaxial Stretch

The “primed” hiPSC-VSMCs expanded in DMEM containing 10% FBS, 2 mM L-glutamine, 1% NEAA, 1 mM sodium pyruvate and 1% pen/strep (or named as Gen 2 hiPSC-VSMCs-10% FBS in FIG. 5) were seeded at 20,000/cm² onto the UNIFLEX™ 6-well culture plates coated with 0.5% (w/v) gelatin (FLEXCELL™ International Corporation) and cultured in the presence of the T/− or T/P TEVG medium for 48-hours. The plates were then either transferred to a FLEXCELL™ FX-6000T™ Tension System (FLEXCELL™ International Corporation) for uniaxial, pulsatile stretching (2.5% strain, 2.75 Hz, sinusoidal waveform, mimicking the stretching regimen for generating TEVG using human primary VSMCs (Dahl et al., 2011, Sci Transl Med 3, 68ra69)), or maintained under static culture for an additional 48-hours. Next, 1 mL medium samples were immediately collected from each well to test the glucose consumption. Additionally, the central rectangular areas of the wells (stretched or static samples) were cut off with a scalpel, and immediately subjected to the subsequent experiments, including the qRT-PCR, immunostaining, hydroxyproline assay, and evaluation of cellular ATP concentration.

### Evaluation of Glucose in Media

The medium samples prior to and after the hiPSC-VSMC culture in the presence or absence of a 48-hour stretching period via a FLEXCELL™ FX-6000T Tension System were collected. The amounts of glucose in media were next evaluated using GLUCCELL™ glucose monitoring system and glucose test strips (CESCO Bioengineering, Inc). The cell numbers in each experimental group were determined by cell counting. To calculate the glucose consumption rates, the changes of amount of glucose during the 48-hour culture period were normalized to the average cell numbers and the duration of culture of each experimental group (2 days). Three biological replicates were completed for evaluation of each experimental group.

### Evaluation of Cellular ATP Concentration

To test the cellular ATP concentrations of hiPSC-VSMCs with or without 48-hour stretching via using FLEXCELL™ FX-6000 Tension System, the total ATP amounts were measured by using the colorimetric ATP assay kit (ABCAM™) according to the instruction. The cell numbers in each experimental group were determined by cell counting using a separated well of hiPSC-VSMCs in the same batch of culture under the same culture condition, and the total ATP amount was normalized to the average cell numbers to derive the cellular ATP concentration of each sample. Three biological replicates were completed for evaluation of each experimental group.

Generation of hiPSC-TEVGs

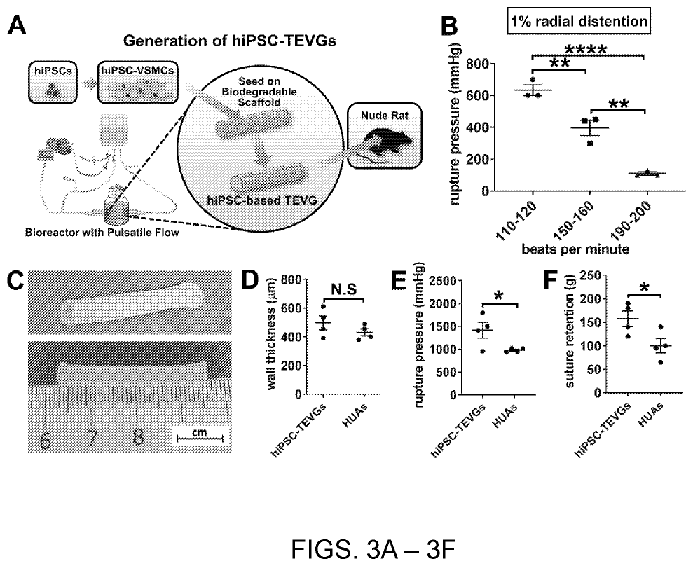

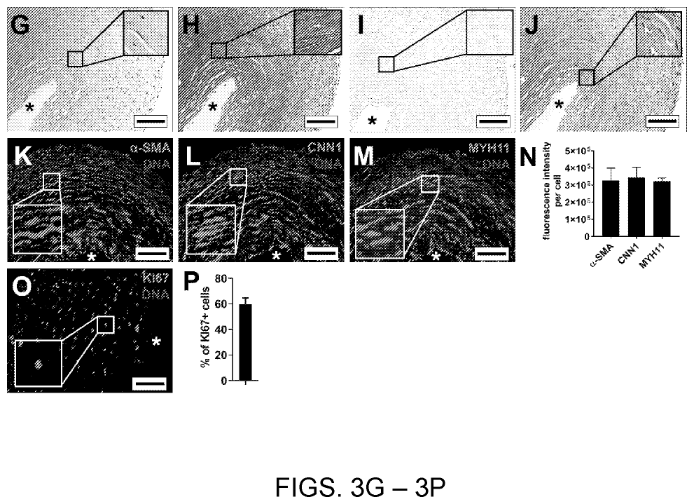

Small-caliber tissue engineered vascular grafts were derived by culturing hiPSC-VSMCs on PGA scaffolds as described previously (Dahl et al., 2011, Sci Transl Med 3, 68ra69; Gui et al., 2016, Biomaterials 102, 120-129; Niklason et al., 1999, Science 284, 489-493). Fifteen million hiPSC-VSMCs were seeded onto tubular, 0.1% gelatin-coated PGA scaffolds which were sewn around silicon tubing (3.2 mm outer diameter; Saint-Gobain) and mounted inside sterilized glass bioreactors (FIG. 3A). Vessels were cultured in 10% FBS expansion medium at 37° C., 5% CO₂ for 24 hours. The expansion medium was then replaced by the optimized TEVG medium (DMEM medium supplemented with 20% FBS, 50 μg/mL ascorbic acid, 50 μg/mL proline, 20 μg/mL alanine, 50 μg/mL glycine, 3 ng/mL CuSO₄, 100 U/ml Penicillin G, 0.13 U/ml insulin and 1 ng/ml TGFβ1). Bioreactor medium (75%) was refreshed every two days. The engineered vessels were cultured without mechanical stimuli for the first week. Thereafter, pulsatile radial stress from a peristaltic pump (COLE-PARMER™) was added incrementally, and the frequency of the mechanical stimuli was adjusted via the rotating speed of the peristaltic pump. The strain induced by the pulsatile radial stress started from 0.5% and was progressively increased to the desired maximum strain at the end of week 4 according to the experiment design. To generate the hiPSC-TEVGs with optimal mechanical strength, 3% of maximum strain was applied, and maintained at the maximum (3%) during the following 4 weeks of culture. By the end of week 8, hiPSC-TEVGs were harvested under sterile conditions for implantation and further analysis.

Implantation of hiPSC-TEVG into Nude Rat

hiPSC-TEVG segments were implanted as abdominal aorta interpositional grafts into 10-week-old, male NIH-Foxn1rnu nude rats (around 300 grams) (Charles River Laboratories). Nude rats were anesthetized with isoflurane, and subsequently opened under standard sterile conditions via a midline abdominal incision leaving the infrarenal abdominal aorta exposed. The cross-clamps were then applied to aid the removal of an aortic segment between the renal artery and the iliac artery. A hiPSC-TEVG segment (7-10 mm in length) was next implanted into the aorta in the “end-to-end” manner using a 10-0 monofilament nylon suture. After confirmation of blood flow and hemostasis following removal of the clamp, the wound area was closed. The animals were left to recover from surgery and maintained for 30 days or 60 days after surgery.

### Ultrasonographical Assessment of Grafts

Nude rats were examined using a Vevo 770® Micro-ultrasound System (VISUALSONICS™, Toronto, Canada) equipped with the RMV-704 scanhead (spatial resolution 40 mm) to determine patency and morphometry of the implanted hiPSC-TEVGs. The inner diameters and outer diameters of the grafts at the midpoint were measured from both transverse and longitudinal axis ultrasound images. The lengths of the grafts were measured from longitudinal axis ultrasound images. The average wall thickness of the midpoint of the implanted grafts was calculated as half of the difference between the outer and inner diameters. For TEVG 1-6, the implanted hiPSC-TEVGs were ultrasonographically analyzed on Day 7, Day 14, Day 21 and Day 28 post-implantation and explanted for histological analysis on Day 30. For TEVG 7, the graft was ultrasonographically analyzed on Day 7, Day 14, Day 21, Day 28, Day 35, Day 42, Day 49 and Day 56 post-implantation and explanted for histological analysis on Day 60.

Mechanical Evaluation of hiPSC-TEVGs

The suture retention strength and rupture pressure of hiPSC-TEVGs were determined as previously described (Gui et al., 2016, Biomaterials 102, 120-129), and HUAs were utilized as a control. Suture retention strength was evaluated by adding weights on a loop of 6-0 Prolene suture (Ethicon) threaded through one side of the TEVG wall, 2 mm from the end, with force applied axially to the graft. The weights were augmented in 10 g to 20 g increments until failure. Four biological replicates were completed for either hiPSC-TEVG or HUA. To measure rupture pressure, vessel segments of 1-1.5 cm long were connected to a flow system coupled with a pressure transducer. DPBS was injected into the flow system until vessel rupture. Four biological replicates were completed for either hiPSC-TEVG or HUA.

The maximum modulus, maximum tensile stress and failure strain of pre-implanted and explanted hiPSC-TEVGs were analyzed using an Instron 5960 microtester (Instron) equipped with a 10 N load cell as previously described (Luo et al., 2017, Biomaterials 147, 116-132). The porcine coronary artery segments with inner diameters of approximately 3 mm were employed as control. The hiPSC-TEVGs or porcine coronary arteries were cut into vessel rings with 1-2 mm in length. The vessel rings were mounted between two stainless steel pins, with one anchored to actuator and the other to the load cell. Vessel rings were next cyclically pre-stretched for three cycles to 10% strain and then increasingly stretched until failure to determine the ultimate tensile strength. Tissue stress was quantified by normalizing tensile force to total cross-sectional area (A=2*π*r²; supposing ring cross section to be circular, r is half of the ring thickness, and cross-sectional area of the ring is multiplied by two to include both sides of the ring), and then the maximum stress, failure strain, maximum modulus of the rings were calculated and plotted. Three independent batches of pre-implanted hiPSC-TEVGs, explanted hiPSC-TEVGs or porcine coronary arteries were subjected to mechanical property evaluation and analyzed.

Contractility Evaluation of hiPSC-TEVGs

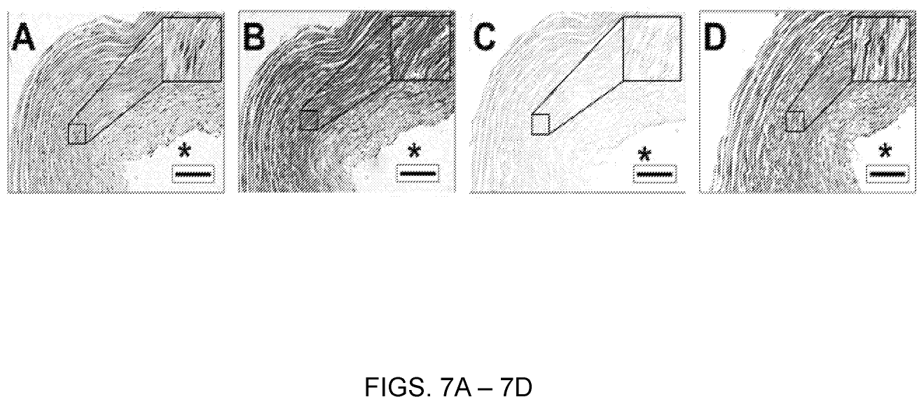

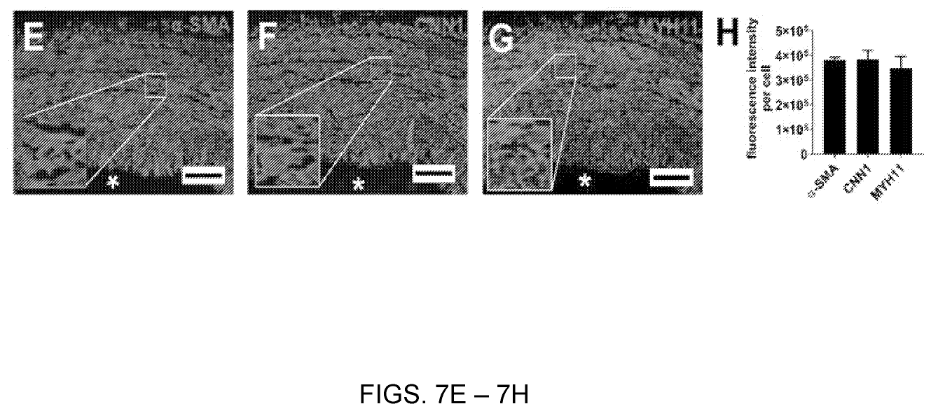

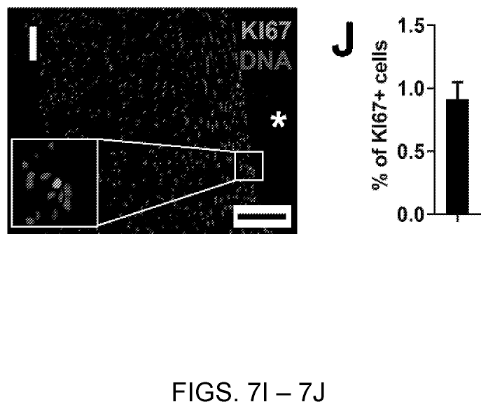

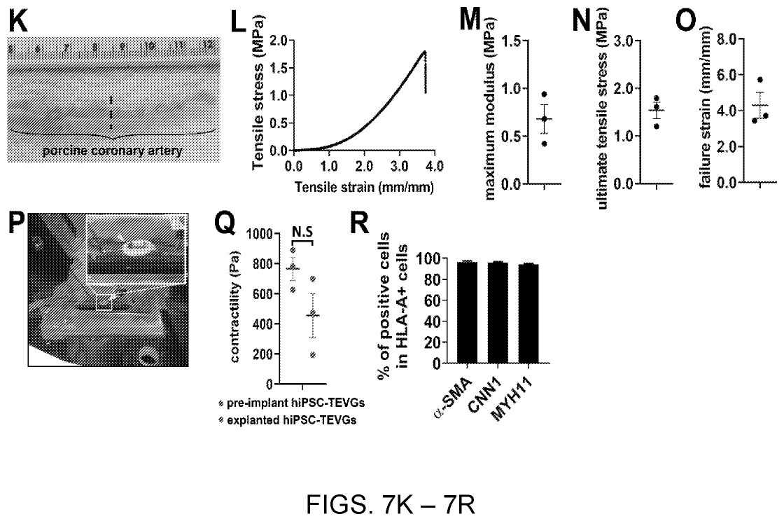

The contractility of the hiPSC-TEVGs was measured as previously described (Luo et al., 2017, Biomaterials 147, 116-132). Briefly, the vessel rings (1-2 mm in length) of pre-implanted or explanted hiPSC-TEVGs 30 days after implantation were sectioned and transferred into a temperature-controlled perfusion bath as shown in FIG. 7P. Vessel rings were hooked between two motorized micromanipulators, which kept the ring suspended between an anchoring point and a force transducer (KG7, SI Heidelberg). When measuring the force, rings were placed in freshly bubbled Tyrode's solution (NaCl 140 mM, KCl 5.4 mM, MgCl₂ 1 mM, HEPES 25 mM, glucose 10 mM and CaCl₂ 1.8 mM, pH 7.3, all from SIGMA-ALDRICH™) at 37° C. Force were measured at the original length, and the manipulators were then moved apart 1.5 mm to evaluate the second reference force prior to stimulation of agonist. The vessel rings were kept untreated for one minute to achieve a baseline value for force change, and then carbachol solution was immediately added to the bath at 1 mM to induce contraction of the ring. The force measurements of vessel rings were recorded for 30 minutes using a Customized MATLAB™ software (MATHWORKS™). The ultimate changes in tension (Pa) were quantified by normalizing the force by the cross-sectional area. The areas of cross-section were derived using optical coherence tomography (Ganymede-II-HR, THORLABS™). An index of refraction of 1.38 was employed for each vessel ring. Images were taken, and their cross-sectional area was quantified by using the NIH Image J software and averaged to calculate the overall cross-sectional area of the vessel rings. Three independent batches of pre-implanted hiPSC-TEVGs or explanted hiPSCs were subjected to contractility tests and analyzed.

### Quantification and Statistical Analysis

All graphic illustrations and statistical analyses were completed using GRAPHPAD™ Prism 6. One-way or two-way ANOVA followed by Tukey post-hoc test was applied for comparison among multiple groups when appropriate. Two-tailed Student's T-test was used to determine the significance of difference between the controls and the experimental groups. A p-value lower than 0.05 was considered significant. Numerical data were reported in format of mean±S.E.M from at least three or more independent experiments. The sample size (n) for each analysis stands for the number of biological replicates and can be found in the figure legends. The statistical details of each experiment can also be found in figure legends and related results.

### Results

Robust, Large-Scale Generation of Functional hiPSC-VSMCs

Generation of hiPSC-TEVGs requires the large-scale production of hiPSC-VSMCs (FIG. 1). A method of deriving hiPSC-VSMCs was first optimized (FIG. 5A). In comparison with previous dispase-assisted embryoid body (EB) formation, more uniform EBs with healthier morphology were derived by EDTA-mediated hiPSC dissociation coupled with an extended medium transition from mTeSR1 (iPSC self-renewal medium) to EB differentiation medium (FIG. 5B; details see Methods and Methods herein). After 7-10 days culture in SmGM-2 (VSMC growth medium), hiPSC-VSMCs in a proliferative state were derived (hiPSC-VSMCs-P). To validate VSMC properties, hiPSC-VSMCs-P were induced into a mature phenotype (hiPSC-VSMCs-M) by a maturation medium containing 1% fetal bovine serum (FBS) and 1 ng/ml transforming growth factor beta 1 (TGF-β1). Similar to human primary VSMCs, hiPSC-VSMCs-M exhibited higher expression levels of VSMC markers including α-smooth muscle actin (α-SMA), calponin (CNN1), and smooth muscle myosin heavy chain (MYH11) as well as extracellular matrix (ECM) markers such as collagen type I (COL1) and elastin (ELN), compared with hiPSC-VSMCs-P (FIGS. 5C-5E). hiPSC-VSMCs did not express octamer-binding transcription factor 4 (OCT4), while hiPSCs were OCT4-positive but VSMC marker-negative (FIGS. 5C-5E). In addition, hiPSC-VSMCs following maturation significantly reduced cell surface area in response to vasoconstrictor (carbachol), indicating their functional contractility (FIGS. 5F-5G). As expected, hiPSC-VSMCs-M showed an increased contractility compared with hiPSC-VSMCs-P.

As growth factors like fibroblast growth factor 2 (FGF2) and epidermal growth factor (EGF) in SmGM-2 medium may reduce VSMC contractile function and collagen production (Chen et al., 2016, Sci Rep 6, 33407; Schlumberger et al., 1991, Arterioscler Thromb 11, 1660-1666), both of which are essential for vascular graft engineering, the inventors “primed” hiPSC-VSMCs-P in DMEM containing 10% FBS without exogenous FGF2 or EGF, and named them generation 2 (Gen 2) hiPSC-VSMCs-10% FBS (FIG. 5H). Gen 2 hiPSC-VSMCs-10% FBS showed an enhanced expression of VSMC (α-SMA, CNN1, MYH11 and smoothelin (SMTH)) and ECM (COL1, COL3 and ELN) markers, but a similar growth rate, compared with hiPSC-VSMCs-P, which were named Gen 2 hiPSC-VSMCs-SmGM in FIGS. 5H-5J. In addition, compared with those Gen 1 hiPSC-VSMCs obtained via a previous approach (Gui et al., 2016, Biomaterials 102, 120-129), Gen 2 hiPSC-VSMCs-10% FBS, based on the currently optimized approach, exhibited a higher proliferative rate as well as an enhanced expression of VSMC (α-SMA, CNN1, MYH11 and SMTH) and ECM (COL1, COL3 and ELN) markers (FIG. 5H-5J). Thus, the inventors elected to use Gen 2 hiPSC-VSMCs-10% FBS (“primed hiPSC-VSMCs”) in subsequent TEVG studies. Routinely, 181.8±10.3 million (n=5) VSMCs from ˜4.5 million hiPSCs are attained in around four weeks, a nine-fold increase of VSMC yield versus previous approaches (Gui et al., 2016, Biomaterials 102, 120-129).

The potential of lineage-specific hiPSC-VSMCs were also evaluated for vascular engineering (Cheung et al., 2012, Nat Biotechnol 30, 165-173). However, lineage-specific hiPSC-VSMCs appeared to be less proliferative than those derived from the EB-based approach (FIG. 5K). As tissue engineering requires cells to promptly expand and fill the void space in the scaffolds before addition of mechanical stimuli, EB-derived hiPSC-VSMCs were chosen to use for vascular engineering herein. Future efforts are warranted to obtain lineage-specific VSMCs with enhanced proliferative capacity for vessel engineering.

Determination of Culture Medium for hiPSC-TEVGs

The previously used medium for culturing hiPSC-TEVGs contained VSMC lineage-specifying growth factors TGF-β1 and platelet-derived growth factor-BB (PDGF-BB) (Gui et al., 2016, Biomaterials 102, 120-129). However, such medium led to TEVGs with limited mechanical strength. The effects of TGF-β1 and PDGF-BB were next investigated on collagen deposition, cell viability and preservation of VSMC phenotype in hiPSC-VSMCs grown on biodegradable PGA scaffolds. The “primed” hiPSC-VSMCs were seeded onto PGA meshes (5 mm×5 mm) and cultured in TEVG medium containing TGF-β1 and PDGF-BB (T/P), TGF-β1 only (T/−), PDGF-BB only (−/P) or no growth factor (−/−), respectively, for three weeks to form engineered tissues (FIG. 2A). Cells effectively populated tissues under all four medium conditions, and the tissues cultured in T/P and T/− media appeared to deposit more collagen than those grown in −/P and −/− media (FIGS. 2B-2C). Hydroxyproline assay further confirmed that hiPSC-VSMCs produced more collagen per mesh in T/P and T/− media (FIG. 2D). T/P and T/− media also appeared to induce higher expression of VSMC markers (α-SMA and MYH11) in hiPSC-VSMCs (FIGS. 2E and 5L). These results suggest that TGF-131 promoted collagen production and VSMC marker expression in hiPSC-VSMCs.

Moreover, engineered tissues in T/P or −/P medium presented a higher ratio of apoptotic cells (FIGS. 2F-2G), suggesting that PDGF-BB may decrease the survival of hiPSC-VSMCs cultured on PGA scaffolds. Thus, a TEVG medium containing TGF-β1 without PDGF-BB (T/−) may provide optimal support for collagen production, cell viability, and maintenance of VSMC properties of hiPSC-VSMCs. In addition, hiPSC-VSMCs in T/− medium readily contracted when exposed to carbachol (FIGS. 5M-5N). Based on the above results, the T/− medium was selected to focus on in the vessel engineering studies.

Generation and Characterization of hiPSC-TEVGs

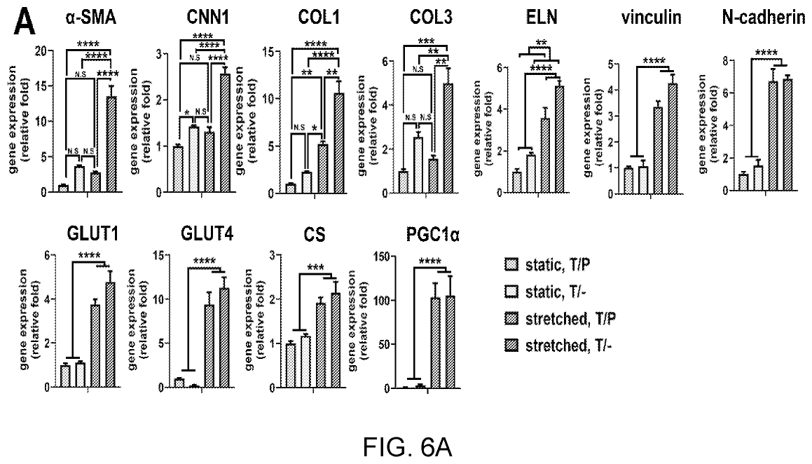

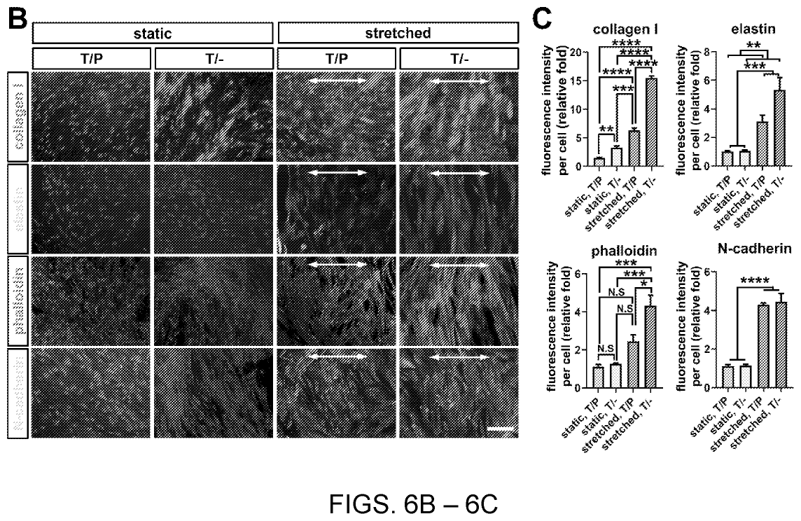

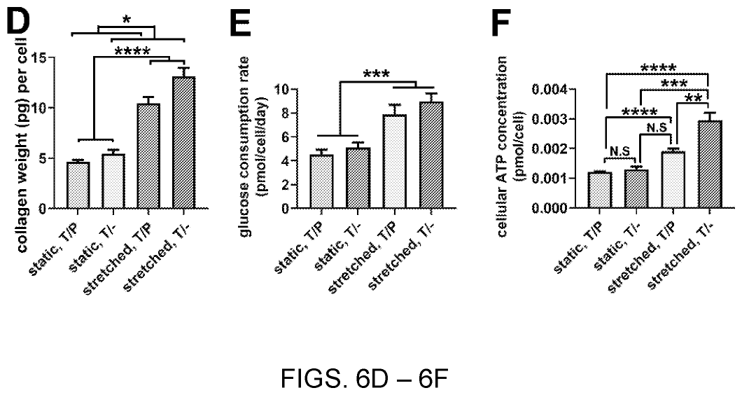

As biomechanical interventions are reported to enhance the strength of engineered tissue constructs (Dahl et al., 2011, Sci Transl Med 3, 68ra69; Syedain et al., 2011, Biomaterials 32, 714-722), the effect of the improved TEVG medium (T/−) on the ECM production and cytoskeletal and metabolic alterations of hiPSC-VSMCs under a work-load via mechanical stretching was investigated. hiPSC-VSMCs were cultured in the presence of the T/− or T/P medium under static or uniaxial cyclic stretching (2.5% distention at 2.75 Hz) with a FLEXCELL™ FX-6000T™ Tension System. This stretching regimen was chosen based on its mechanical enhancement of the previously reported, primary VSMC-based TEVGs (Dahl et al., 2011, Sci Transl Med 3, 68ra69). Results suggest that both T/− medium and a 48-hour stretching period enhanced the expression of VSMC (α-SMA and CNN1) and ECM (COL1, COL3, and ELN) markers (FIGS. 6A-6C). Hydroxyproline assay further revealed that both T/− medium and stretching promoted collagen deposition in hiPSC-VSMCs (FIG. 6D). Furthermore, hiPSC-VSMCs cultured in T/− medium under stretching appeared to have enhanced formation of filamentous actin bundles (phalloidin) with a preferred alignment perpendicular to the direction of stretching (FIGS. 6B-6C). Moreover, stretching alone increased the expression of focal adhesion (vinculin) or adherens junction (N-cadherin) markers in hiPSC-VSMCs (FIGS. 6A-6C).

Mechanical stretching also resulted in an increase in energy production. Since hiPSC-VSMCs were cultured in a TEVG medium based on high-glucose DMEM basal medium (˜25 mM glucose), glucose was hypothesized to be the predominant energy source and further investigated. The results showed that stretching increased glucose consumption and cellular ATP production (FIGS. 6E-6F). When coupled with the optimized medium (T/−), there was an even further increase in cellular ATP production (FIG. 6F). These findings were also reflected transcriptionally with significant upregulation of pivotal glucose metabolism-associated genes under stretching, including the glucose transporters-1 and -4 (GLUT1 and GLUT4), citrate synthase (CS; involved in mitochondrial TCA cycle metabolism), and peroxisome proliferator-activated receptor gamma coactivator 1-alpha (PGC1α; a transcriptional cofactor of mitochondrial biogenesis) (FIG. 6A), suggesting a degree of metabolic maturation was induced in these hiPSC-VSMCs. In summary, the optimized T/− TEVG medium and mechanical stretching effectively enhanced VSMC marker expression, ECM deposition, cytoskeleton alignment, cellular adhesion, and ATP production in the hiPSC-VSMCs. These results suggest that both mechanical stretching and an optimized TEVG medium would potentially promote the generation of hiPSC-based TEVGs with advanced mechanical strength.

Then TEVGs were fabricated from hiPSC-VSMCs and augmented their mechanical strength with the inclusion of pulsatile radial stretching. As illustrated in FIG. 3A, PGA scaffolds were sewn over silicone tubing (˜3.2 mm outer diameter) and connected in bioreactors. Scaffolds were seeded with hiPSC-VSMCs and cultured statically for one week. Dulbecco's phosphate buffered saline (DPBS) was then flowed by a peristaltic pump into distensible silicone tubing inserted through the vessel lumen, and pulsatile radial stress was applied to the vessels for seven weeks. Radial stress with 1-2.5% radial distention (strain) has been applied to develop TEVGs from human primary VSMCs (Dahl et al., 2011 Sci Transl Med 3, 68ra69; Poh et al., 2005, Lancet 365, 2122-2124). However, acute addition of 2% strain repeatedly led to the disintegration of the engineered tissue after three weeks of culture, suggesting that abrupt exposure to this level of mechanical stress may be detrimental to hiPSC-VSMCs. This phenomenon prompted us to add radial stress incrementally from week two to week four, and then maintain the strain unchanged until the completion of culture. Using this novel mechanical regimen, hiPSC-TEVGs successfully maintained integrity during the eight-week culture. Previous studies revealed a pulse rate (beats per minute, bpm) of radial distention at ˜165 bpm resulted in optimal mechanical strength of human or porcine primary VSMC-derived TEVGs (Dahl et al., 2011, Sci Transl Med 3, 68ra69; Solan et al., 2009, Cell Transplant 18, 915-921). The rupture pressure was evaluated of hiPSC-TEVGs developed in the presence of 1% radial distention at several pulse rates (110-120 bpm, 150-160 bpm, and 190-200 bpm). Surprisingly, distinct from human primary VSMCs, 110-120 bpm resulted in the highest rupture pressure of hiPSC-TEVGs, and rupture pressure declined dramatically along with an increase in bpm (FIG. 3B). Thus, pulsatile radial stress of 110-120 bpm was applied in the hiPSC-TEVGs.

Next, the ultimate strain was increased to 3% and maintained the pulse rate at 110-120 bpm to generate hiPSC-TEVGs. At the end of the eight-week culture period, hiPSC-TEVGs appeared opaque, similar to native vessels (FIG. 3C). The wall thickness of hiPSC-TEVGs was 496.8±48.0 μm (n=4, FIG. 3D). Importantly, hiPSC-TEVGs showed robust mechanical properties indicated by rupture pressure (1419.0±174.4 mmHg, n=4) and suture retention strength (157.5±16.5 g, n=4) (FIGS. 3E and 3F) comparable to those of native vessels widely used as coronary artery bypass grafts (saphenous veins, rupture pressure: 1599±877 mmHg; suture retention strength: 196±29 g) (Dahl et al., 2011, Sci Transl Med 3, 68ra69). Moreover, the mechanical strength of the hiPSC-TEVGs was markedly higher than that of native human umbilical arteries (HUA, rupture pressure: 930.4±23.3 mmHg, n=4; suture retention strength: 102.5±17.6 g, n=4), while the thickness (HUA, 431.25±25.20 μm, n=4) was similar (FIGS. 3D-3F). In addition, the amount of collagen in hiPSC-TEVGs was 43.1±4.4% (n=4) of the dry weight of the engineered vessels, which was comparable to that of primary human VSMC-derived TEVGs (31±7%) (Quint et al., 2012, J Vasc Surg 55, 790-798) or native blood vessels (HUA, 36±12%) (Gui et al., 2016, Biomaterials 102, 120-129). In comparison to the mechanical properties of hiPSC-TEVGs previously developed without pulsatile radial stress (rupture pressure: 500 mmHg, n=2; suture retention strength: 30 and 70 g, n=2; collagen amount: 12±4% of the dry weight of engineered vessels, n=2) (Gui et al., 2016, Biomaterials 102, 120-129), the current results revealed that the addition of novel incremental pulsatile radial stretching coupled with the optimized TEVG medium markedly improved the biomechanical properties of hiPSC-TEVGs.

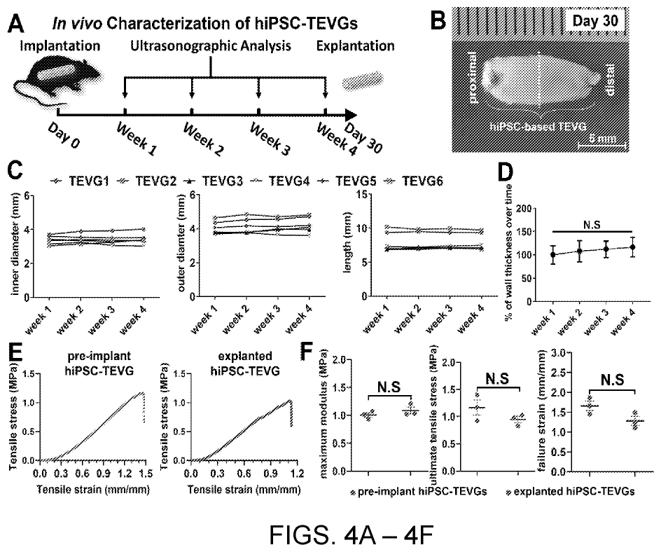

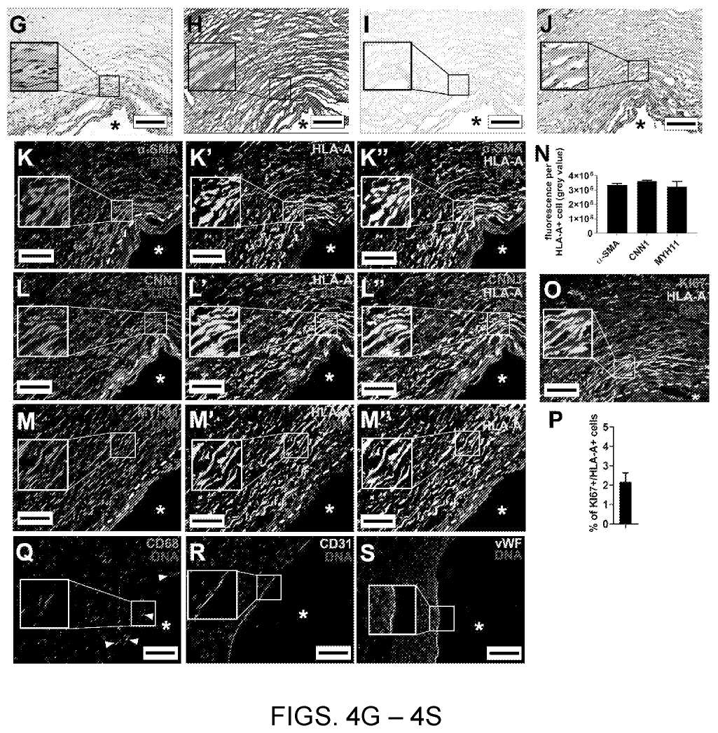

Similar to a human native blood vessel (HUA, FIGS. 7A-7J), histological analysis showed hiPSC-TEVGs were highly cellularized (FIG. 3G), exhibited robust accretion of collagen (FIG. 3H), and lacked obvious calcium deposition (FIG. 3I). Similar to previous TEVGs derived from primary or hiPSC-VSMCs (Gui et al., 2016; Niklason et al., 1999), mature ELN fibers were absent in hiPSC-TEVGs (FIG. 3J). Future efforts are warranted to optimize TEVG culture to enhance ELN function. PGA remnants were seldom observed in hiPSC-TEVGs (FIGS. 3G-3J), indicating thorough scaffold degradation. Typical VSMC markers (α-SMA, CNN1 and MYH11) were present in the cells within the hiPSC-TEVGs (FIGS. 3K-3N), similar to those expressed within the native HUAs (FIGS. 7E-7H). Interestingly, contrary to the minimal number of cells with positivity for the proliferation marker KI67 in native HUAs (0.91±0.13%, FIGS. 7I-7J), a significant number of cells in the hiPSC-TEVGs were positive for KI67 (59.79±4.86%, FIG. 3O-3P), potentially due to the cell growth-promoting nature of the TEVG medium. Importantly, cells in hiPSC-TEVGs became largely quiescent under physiological conditions after in vivo implantation into the rat abdominal aorta (FIGS. 4O-4P). These data suggest that hiPSC-TEVGs displayed the typical histological and cellular phenotypes of engineered vessels.

Implantation of hiPSC-TEVGs into Nude Rats

Six hiPSC-TEVGs (TEVG 1-6) were then implanted, as an interpositional graft, into the abdominal aorta of six nude rats (FIG. 4A). The nude rat model was employed to avoid overt immunorejection against implanted human tissue. As this was a proof-of-principle study to evaluate the mechanical properties of hiPSC-TEVGs after exposure to the rat aortic hemodynamics in vivo, the hiPSC-TEVGs were not supplemented with a luminal endothelial layer. All rats were followed for 30 days, and the morphometry of implanted hiPSC-TEVGs (mid-graft inner diameter, mid-graft outer diameter and length) were assessed weekly via ultrasonography (FIG. 4A). All implanted grafts were patent when harvested, and no sign of rupture or aberrant deformation was observed in any of the explants (FIG. 4B), potentially due to the high mechanical strength of the grafts (FIG. 3E-3F). No sign of teratoma formation was observed in any animals. Ultrasonography revealed that the grafts did not present any evident dilation, elongation or wall thickening (FIG. 4C-4D). Limited thrombosis was observed at distal ends of some TEVGs, possibly due to the lack of an endothelial lining prior to graft implantation, and/or the potential intraluminal turbulence of blood flow. These results indicated that the hiPSC-TEVGs were able to withstand aortic blood pressure during the period of evaluation.

Next, the mechanical properties of the explanted hiPSC-TEVGs (TEVG 4-6) were examined by cutting them into tissue rings/strips and deriving stress-strain plots (representative plots shown in FIG. 4E) using the INSTRON™ microtester system. Mechanical parameters such as maximum modulus (the maximum slope of the linear region of the curve), ultimate tensile stress, and failure strain were analyzed. As a reference, tissue rings made from the same hiPSC-TEVG prior to implantation were also measured for comparison. As shown in FIGS. 4E-4F, the mechanical properties, including maximum modulus, ultimate tensile stress, and failure strain of tissue rings derived from hiPSC-TEVGs pre- and post-implantation appeared to be statistically comparable (FIG. 4F). Tissue rings derived from porcine native coronary arteries with comparable inner diameter were used as a control (FIG. 7K). The porcine arterial rings showed a higher failure strain, and a trend towards an enhanced ultimate tensile stress (p=0.052, one-way ANOVA) and a reduced maximum modulus (p=0.054, one-way ANOVA) compared with the hiPSC-TEVG counterparts (FIG. 7L-7O), potentially due to the more effective accumulation of ELN and collagen deposition in the porcine native vessels. The contractile function of the tissue rings sectioned from hiPSC-TEVGs was also evaluated in the presence of the vasoconstrictor carbachol, based on direct force measurement by a force transducer (FIG. 7P) (Luo et al., 2017, Biomaterials 147, 116-132). No statistically significant difference was detected between the contractility of pre-implant and explanted hiPSC-TEVGs in response to a 30-minute carbachol treatment (FIG. 7Q). There appeared to be a trend of decreased contractility in the explanted tissue rings without reaching significance (p=0.13), possibly due to suboptimal luminal lining and modeling in vivo, as a luminal endothelial layer was not supplemented prior to implantation in this proof-of-principle study. In summary, these results suggest that hiPSC-TEVGs maintained mechanical strength and contractile function after implantation.

Histological analysis revealed that explanted grafts remained cellularized (FIG. 4G), that extracellular collagen was abundantly deposited (FIG. 4H), and there was no apparent calcification in the media of the grafts (FIG. 4I). Mature ELN fibers were not detected (FIG. 4J), similar to previously reported TEVGs derived from hiPSC-VSMCs or primary VSMCs (Gui et al., 2016, Biomaterials 102, 120-129; Niklason et al., 1999, Science 284, 489-493). VSMC markers (α-SMA, CNN1 and MYH11) were readily observed in human cells (positive for human HLA-A staining, above the white dashed line in FIG. 4K-4M and FIG. 7R) within the grafts. Additionally, expression levels of α-SMA, CNN1 and MYH11 in hiPSC-VSMCs in explanted TEVGs (FIG. 4N) appeared to be comparable to those of the respective VSMC markers in the hiPSC-VSMCs in pre-implant grafts (FIG. 3N). A minimal number of human cells in the grafts were positive for proliferation marker KI67 (FIG. 4O-4P), suggesting that hiPSC-derived cells within the vessel walls were primarily in a quiescent state, which was similar to cells in the human native HUAs (FIG. 7I-7J). In addition, only very limited numbers of cluster of differentiation 68+ (CD68+) cells were observed near the luminal side, suggesting minimal or no presence of macrophages in the grafts (FIG. 4Q). Notably, some portions of the luminal surface displayed coverage by endothelial cells from the host, revealed by CD31 and von Willebrand factor (vWF) staining (FIG. 4R-4S). In summary, these data suggest that hiPSC-derived cells in the engineered vessel preserved their mature VSMC phenotype following in vivo implantation.

Decellularization of hiPSC-TEVGs and hiPSC-EC Endothelialization

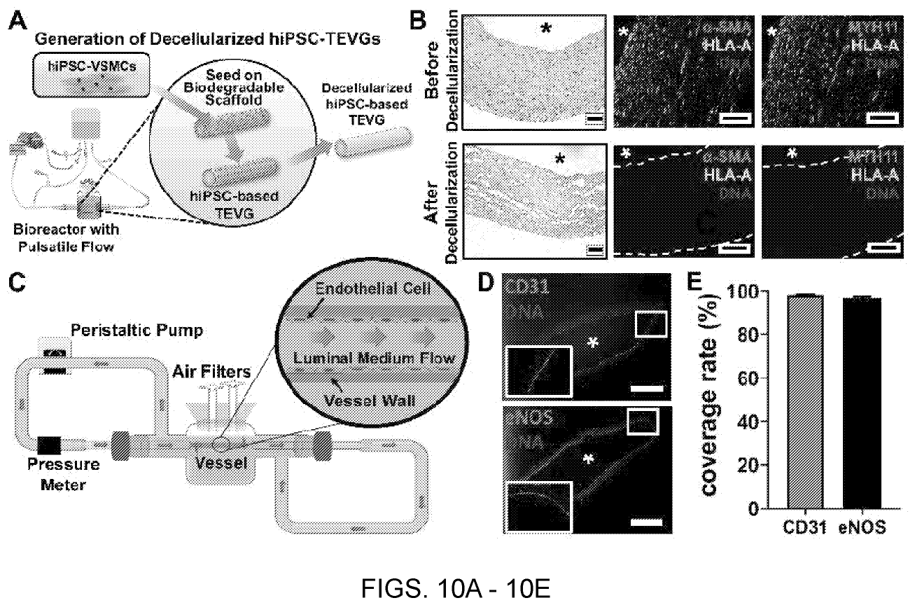

Human induced pluripotent stem cell-derived tissue engineered vascular grafts (hiPSC-TEVGs) were decellularized. Briefly, TEVGs were placed into a decellularization solution (8 mM CHAPS, 1 M NaCl, and 25 mM EDTA in PBS), and stirred for 24 hours in an incubator at 37° C. The vessels were then rinsed in PBS, and placed in a second decellularization solution (1.8 mM SDS, 1 M NaCl, and 25 mM EDTA in PBS), followed by 24 hours stirring at 37° C. and then rinsing in PBS. TEVGs were stored in PBS at 4° C. after decellularization. H&E, α-SMA, MYH11, HLA-A, and DNA staining revealed effective decellularization of TEVGs (FIGS. 10A-10B). Acellular grafts possessed similar mechanical strength: suture retention (125.7±10.7 g, n=4) and rupture pressure (1259.5±151.5 mmHg, n=2), to those prior to decellularization. Next, wild type hiPSC-derived endothelial cells (hiPSC-ECs) were used to endothelialize decellularized hiPSC-TEVGs. The vessel lumen was coated with human fibronectin (50 μg/ml) followed by two to three rounds of 1-hour seeding with hiPSC-ECs (1.5×10⁶/cm²). Shear stress was then started at 1 dyne/cm² and progressively increased to 15 dynes/cm² during a 3.5-day period (FIG. 10C). The vessel was next kept at 15 dynes/cm² for 1.5 day. Immunostaining of vessel sections for EC markers (CD31 and eNOS) revealed effective EC coverage (98.3±0.3% and 96.2±1.5%) (FIGS. 10D-10E).

Generation of Hypoimmunogenic, Universal hiPSC-ECs

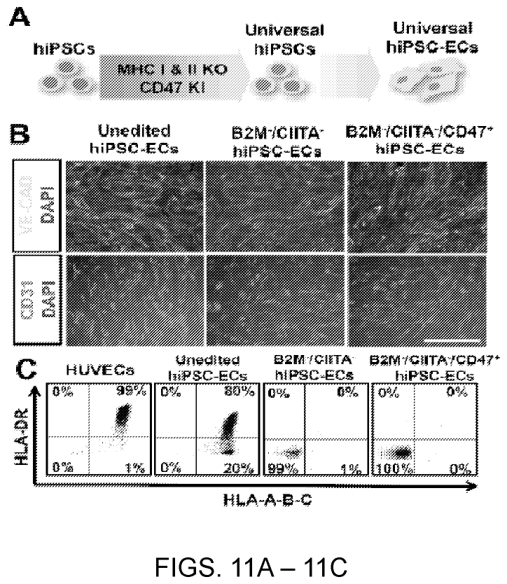

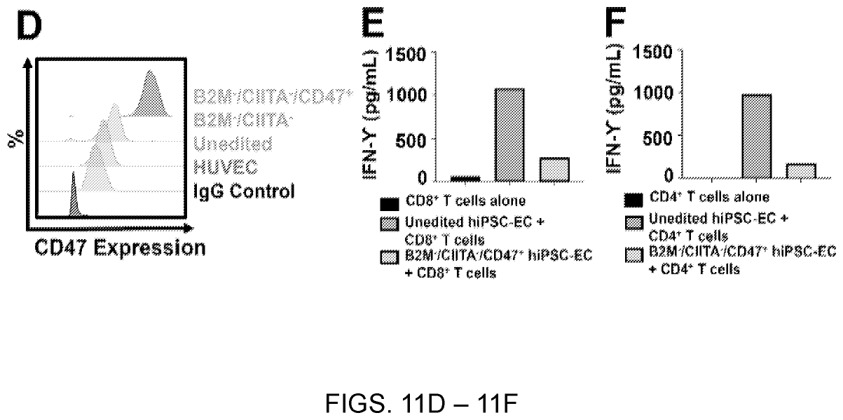

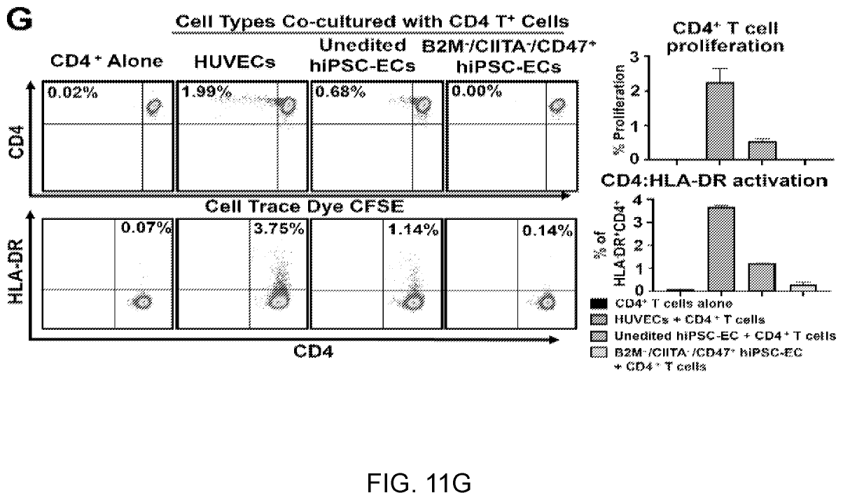

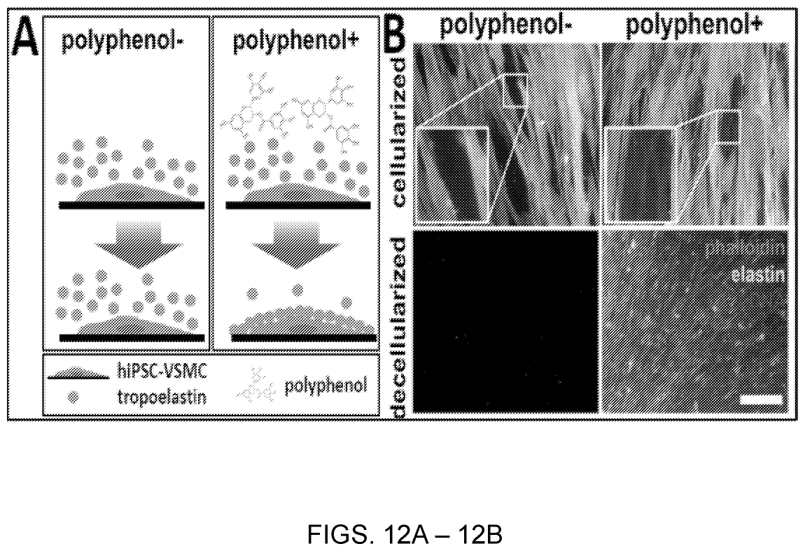

To obtain hypoimmunogenic universal hiPSCs, a CRISPR-Cas9 approach was used for inactivating the coding sequence (exon 2) of the human β2-microglobulin (B2M) gene, a common component of MHC class I molecules, as well as the coding sequence (exon 3) of the human CIITA gene, the master regulator of MHC class II molecules, paired with an ectopic expression of CD47 TALEN-mediated insertion at the AAVSI “safe harbor” gene locus (FIG. 11A). These gene edited hiPSCs were then differentiated into ECs, and these ECs presented expression of typical EC markers (CD31 and VE-cadherin, FIG. 11B). Compared with the wild type human primary ECs (HUVECs) and unedited hiPSC-ECs, both B2M⁻/CIITA⁻ and B2M⁻/CIITA⁻/CD47⁺ hiPSC-ECs displayed minimal expression of MHC molecules when treated with interferon gamma (IFN-γ) (FIG. 11C), and B2M⁻/CIITA⁻/CD47⁺ hiPSC-ECs additionally expressed high levels of CD47 (FIG. 11D). Next, we co-cultured these edited hiPSC-ECs with human CD4+ and CD8+ effector memory T-cells (Tem). Data suggested that both CD4+ and CD8+ Tem cells secreted markedly lower levels of IFN-γ when co-cultured with B2M⁻/CIITA⁻/CD47⁺ universal hiPSC-ECs, compared to those cultured with unedited counterparts (FIG. 11E-11F). Moreover, CD4+ Tem cells co-cultured with B2M⁻/CIITA⁻/CD47⁺ hiPSC-ECs exhibited a reduction in cell proliferation and activation marker expression compared with the co-culture controls with unedited hiPSC-ECs (FIG. 11G).

### Enhancement of Elastin (ELN) Deposition by Polyphenol Treatment

Polyphenols, such as epigallocatechin gallate (EGCG), could enhance ELN deposition in rat primary VSMCs via increasing the assembly of monomeric tropoelastin (FIG. 12A) (Sinha et al., 2014, Biochem Biophys Res Commun., 444, 205-211). hiPSC-VSMCs were cultured in 3% FBS containing 1 ng/ml TGF-β1 and observed promising ELN fiber formation (FIG. 12B). Further optimization of this medium to increase ELN deposition in hiPSC-TEVGs is ongoing.

### Discussion

The results described herein represent the most mechanically robust derivation of TEVGs from hiPSC-VSMCs in the field, with properties approaching those of native vessels used for arterial bypass. The routine, robust production (˜180 million) of functional VSMCs from self-renewable hiPSCs provides an unlimited supply of VSMCs for vascular engineering. This, coupled with an optimized TEVG culture medium containing TGF-β1 but without PDGF-BB, and a novel biophysical training regimen of hiPSC-VSMC-derived TEVGs in a bioreactor, including an incremental, radial stretching regimen and an efficacious pulse rate of radial distention at 110-120 bpm, markedly enhanced the mechanical properties of hiPSC-TEVGs. Importantly, hiPSC-TEVGs showed excellent patency without radial dilation or longitudinal elongation and effectively maintained both mechanical and contractile function four weeks after abdominal aortic implantation in rats. This study has thus established the foundation for large-scale manufacture of mechanically robust hiPSC-TEVGs as a novel therapy (FIG. 1B-C).

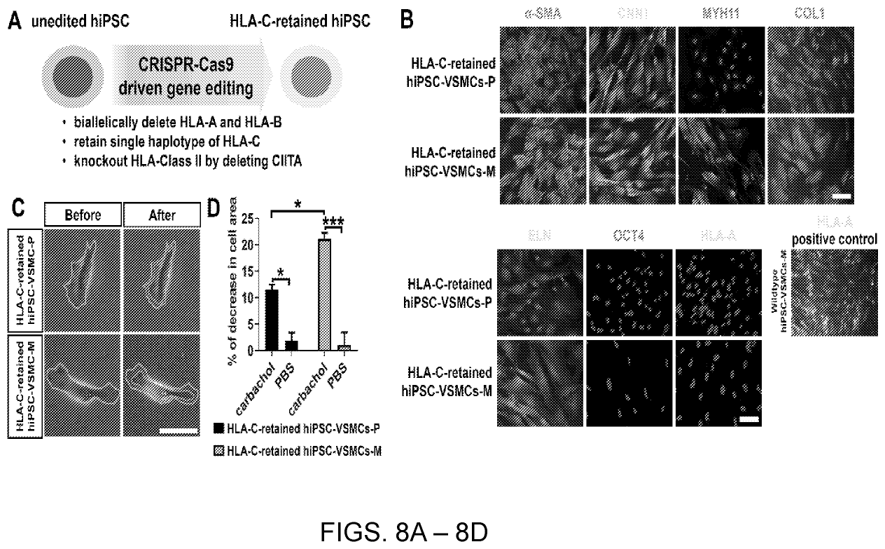

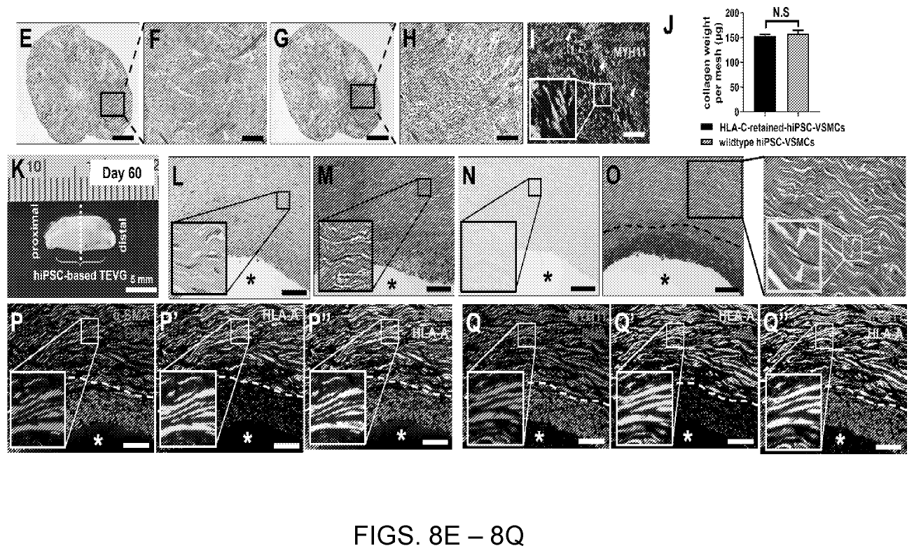

Additionally, this technology could integrate immunocompatible, HLA-engineered universal hiPSC lines (Deuse et al., 2019, Nat Biotechnol 37, 252-258; Gornalusse et al., 2017, Nat Biotechnol 35, 765-772; Xu et al., 2019, Cell Stem Cell 24, 566-578 e567) in the future for allogeneic graft implantation as a treatment option for any patient in need (FIG. 1B). As a proof-of-concept exploration, VSMCs were derived from HLA-C-retained hiPSCs with enhanced immunocompatibility, in which both HLA-A and -B alleles and HLA-class II are disrupted (FIG. 8A) (Xu et al., 2019, Cell Stem Cell 24, 566-578 e567). It is estimated that 12 HLA-C-retained iPSC lines are immunocompatible with >90% of the world's population, significantly facilitating iPSC-based therapies. HLA-C-retained hiPSC-VSMCs expressed VSMC (α-SMA, CNN1 and MYH11) and ECM (COL I and ELN) markers (FIG. 8B), showed contraction in response to carbachol (FIG. 8C-8D), and readily formed vascular tissue on PGA scaffolds with effective collagen production (FIGS. 8E-8J), suggesting the potential feasibility of vascular tissue engineering using HLA-edited, immunocompatible hiPSC lines in the future.

Moreover, as an attempt to observe ELN formation in a longer term in vivo, a 60-day rat aortic implantation of hiPSC-TEVG was performed. The graft remained patent with effective cellularity and collagen deposition and without discernable calcification (FIG. 8K-Q). Although appreciable mature ELN fibers were not observed in the medial layers, limited extracellularly deposited, discontinuous ELN fibers were detected in the two-month explanted graft via EVG staining (FIG. 8O).

Further, a hypoimmunogenic universal hiPSC line has been developed by modulating the expression of human leukocyte antigens (HLAs) and endothelial cells (ECs) have been derived from this line. Moreover, hiPSC-TEVGs have been decellularized and endothelialized with hypoimmunogenic, universal ECs. This invention thus lays the foundation for the fabrication of readily available, small caliber hiPSC-TEVGs containing functional endothelium immunocompatible to any patient recipient, potentially transforming clinical vascular tissue engineering.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A tissue-engineering vascular graft (TEVG) comprising:
a biodegradable scaffold, and
a plurality of stem cell-derived vascular smooth muscle cells (VSMCs),
wherein the plurality of stem cell-derived VSMCs are seeded on the biodegradable synthetic polymer scaffold and are cultured under mechanical and biochemical stimulation.

2. The TEVG according to claim 1, wherein the biodegradable scaffold comprises one or more synthetic polymers selected from: polyglycolic acid (PGA), polylactic acid (PLA), polycaprolactone, polyethylene glycol, polylactic-co-glycolic acid (PLGA), poly(glycerol sebacate) (PGS), fast-degrading polymers, and/or combinations thereof.

3. The TEVG according to claim 1, wherein the stem cell-derived VSMCs are derived from human induced pluripotent stem cells (hiPSCs) that are induced to differentiate into VSMCs.

4. The TEVG according to claim 1, wherein the stem cell-derived VSMCs are allogeneic.

5. The TEVG according to claim 1, further comprising a plurality of stem cell-derived vascular endothelial cells (ECs).

6. The TEVG according to claim 5, wherein the stem cells are hiPSCs.

7. The TEVG according to claim 5, wherein the stem cell-derived ECs are allogeneic.

8. The TEVG according to claim 1, wherein the mechanical stimulation comprises incremental radial stretching and pulsatile radial distension.

9. The TEVG according to claim 8, wherein the pulsatile radial distension has a pulse rate of about 110 to about 120 bpm.

10. The TEVG according to claim 1, wherein the biochemical stimulation comprises TEVG culture media.

11. The TEVG according to claim 10, wherein the TEVG culture media comprising transforming growth factor-β1 (TGF-β1) and does not comprise platelet-derived growth factor-BB (PDGF-BB).

12. The TEVG according to claim 3, wherein the hiPSCs are immunocompatible pluripotent stem cells.

13. The TEVG according to claim 6, wherein the hiPSCs are immunocompatible pluripotent stem cells.

14. The TEVG according to claim 2, wherein the fast-degrading polymers comprise 87% glycolide, 7% trimethylene carbonate (TMC), and 6% polyethylene glycol.

15. A method of generating a tissue-engineered vascular graft (TEVG), the method comprising:
a) obtaining a plurality of hiPSCs;
b) inducing the plurality of hiPSCs to differentiate into a population of hiPSC-VSMCs;
c) seeding the population of hiPSC-VSMCs onto a biodegradable scaffold; and
d) culturing the population of hiPSC-VSMCs on the biodegradable scaffold under mechanical and biochemical stimulation for a duration of time, thereby generating a hiPSC-TEVG.

16. The method according to claim 15, wherein the hiPSCs are allogeneic.

17. The method according to claim 15, wherein the hiPSCs are autogeneic.

18. The method according to claim 15, wherein the biodegradable scaffold comprises one or more synthetic polymers selected from: polyglycolic acid (PGA), polylactic acid (PLA), polycaprolactone, polyethylene glycol, polylactic-co-glycolic acid (PLGA), poly(glycerol sebacate) (PGS), fast-degrading polymers, and combinations thereof.

19. The method according to claim 15, wherein the mechanical stimulation comprises incremental radial stretching and pulsatile radial distension.

20. The method according to claim 19, wherein the pulsatile radial distension has a pulse rate of about 110 to about 120 bpm.

21. The method according to claim 15, wherein the biochemical stimulation comprises TEVG culture media.

22. The method according to claim 21, wherein the TEVG culture media comprising transforming growth factor-β1 (TGF-β1) and does not comprise platelet-derived growth factor-BB (PDGF-BB).

23. The method according to claim 15, further comprising the intermediate step:
b′) inducing the plurality of hiPSCs to differentiate into a population of hiPSC-derived ECs (hiPSC-EC).

24. The method according to claim 23, further comprising:
e) seeding the hiPSC-TEVG with the population of hiPSC-ECs, thereby endothelializing the TEVG.

25. The method according to claim 15, further comprising the intermediate step of:
a′) modulating the human leukocyte antigen (HLA) expression of the plurality hiPSCs.

26. The method according to claim 18, wherein the fast-degrading polymer comprises 87% glycolide, 7% trimethylene carbonate (TMC), and 6% polyethylene glycol.

27. The method according to claim 15, wherein the population of hiPSC-VSMCs are cultured in media comprising one or more polyphenol.

28. The method according to claim 27, wherein the one or more polyphenols comprise epigallocatechin gallate (EGCG).

29. A tissue-engineering vascular graft (TEVG) comprising:
a biodegradable scaffold, and
a plurality of stem cell-derived vascular smooth muscle cells (VSMCs), and
a plurality of stem cell-derived vascular endothelial cells (ECs),
wherein the plurality of stem cell-derived VSMCs are seeded on the biodegradable synthetic polymer scaffold and are cultured under mechanical and biochemical stimulation.

30. The TEVG of claim 29, wherein the stem cell-derived ECs are allogeneic.

31. The TEVG of claim 30, wherein the stem cell-derived ECs are B2M⁻/CIITA⁻/CD47⁺ hiPSC-derived ECs (hiPSC-ECs).


SEQUENCE LISTING (28 sequences)

SEQ ID NO: 1 - DNA, 21 bp, homo sapiens
tgttgccatcaatgacccctt

SEQ ID NO: 2 - DNA, 19 bp, homo sapiens
ctccacgacgtactcagcg

SEQ ID NO: 3 - DNA, 18 bp, homo sapiens
ctgggacgacatggaaaa

SEQ ID NO: 4 - DNA, 18 bp, homo sapiens
acatggctgggacattga

SEQ ID NO: 5 - DNA, 22 bp, homo sapiens
agcatggcgaagacgaaaggaa

SEQ ID NO: 6 - DNA, 22 bp, homo sapiens
cccatctgcaggctgacattga

SEQ ID NO: 7 - DNA, 23 bp, homo sapiens
agagacagcttcacgagtatgag

SEQ ID NO: 8 - DNA, 22 bp, homo sapiens
cttccagctctctttgaaagtc

SEQ ID NO: 9 - DNA, 20 bp, homo sapiens
cctggatacagaggacatgg

SEQ ID NO: 10 - DNA, 20 bp, homo sapiens
caggtggttgtagagcgact

SEQ ID NO: 11 - DNA, 22 bp, homo sapiens
cctgtctgcttcctgtaaactc

SEQ ID NO: 12 - DNA, 21 bp, homo sapiens
gttcagtttgggttgcttgtc

SEQ ID NO: 13 - DNA, 22 bp, homo sapiens
gctctgcttcatcccactatta

SEQ ID NO: 14 - DNA, 22 bp, homo sapiens
ctggcttccagacatctctatc

SEQ ID NO: 15 - DNA, 20 bp, homo sapiens
aagatggtgcagacacttcc

SEQ ID NO: 16 - DNA, 20 bp, homo sapiens
agagcgaatccagctttgag

SEQ ID NO: 17 - DNA, 20 bp, homo sapiens
aatggtccagcaagggcaat

SEQ ID NO: 18 - DNA, 20 bp, homo sapiens
gaatgagtgcccgcttggta

SEQ ID NO: 19 - DNA, 22 bp, homo sapiens
gggaaatggaaacttgatggca

SEQ ID NO: 20 - DNA, 25 bp, homo sapiens
cagttgctaaacttcactgaaagga

SEQ ID NO: 21 - DNA, 20 bp, homo sapiens
tggcatcaacgctgtcttct

SEQ ID NO: 22 - DNA, 20 bp, homo sapiens
agccaatggtggcatacaca

SEQ ID NO: 23 - DNA, 20 bp, homo sapiens
ggttctttcatcttcgccgc

SEQ ID NO: 24 - DNA, 20 bp, homo sapiens
tccccatcttcggagcctat

SEQ ID NO: 25 - DNA, 20 bp, homo sapiens
ctcaactcaggacgggttgt

SEQ ID NO: 26 - DNA, 20 bp, homo sapiens
ggggtcattaggcaggtgtt

SEQ ID NO: 27 - DNA, 20 bp, homo sapiens
ctacggctcctcctgggaaa

SEQ ID NO: 28 - DNA, 20 bp, homo sapiens
cagtccaggggcagaaaagt